(19) United States
(12) Patent Application Publication
(10) Pub. No.: US 2026/0337600 A1
(43) Pub. Date: Oct. 29, 2026

(54) HIGHLY EFFICIENT METHOD FOR OBTAINING GENE SEQUENCE

(71) Applicant: bitBiome, Inc., Tokyo (JP)

(72) Inventors: Masahito HOSOKAWA, Tokyo (JP); Tatsuya SAEKI, Tokyo (JP); Tariho ENDO, Tokyo (JP); Kazuma KAMATA, Tokyo (JP)

(73) Assignee: bitBiome, Inc., Tokyo (JP)

(21) Appl. No.: 19/118,251

(22) PCT Filed: Oct. 4, 2023
(86) PCT No.: PCT/JP2023/036256

§ 371 Date: Apr. 3, 2025

(30) Foreign Application Priority Data
Oct. 4, 2022 (JP) 2022-160265

(51) Int. Cl. C12N 15/10; G16B 20/00; G16B 50/00

(52) U.S. Cl. CPC C12N 15/1065; G16B 20/00; G16B 50/00

(57) ABSTRACT

The present disclosure provides a novel highly efficient method for obtaining a gene sequence. More specifically, the present disclosure provides a highly efficient method for obtaining a gene sequence using a nucleic acid library containing a nucleic acid derived from one or more cells of cell-like structures and/or an amplification product obtained by whole genome amplification using the nucleic acids as a template, in one small section, and including one or a plurality of small sections; and a library, a system, a program, and the like which can be used in the highly efficient method for obtaining a gene sequence.

### BACKGROUND ART

### Technical Field

Until now, microorganisms have served as sources for many pharmaceutical products including penicillin, streptomycin, leucomycin, mitomycin, pravastatin, ivermectin, tacrolimus, and micafungin, and have also been utilized in the food industry for the production of miso, soy sauce, shochu, and wine. Furthermore, in the environmental field, microorganisms have been used for the purification of contaminated soil. In this manner, humanity has received countless benefits from microorganisms, Many benefits offered by those microorganisms are ultimately realized by various genes encoded in the genome of the microorganisms. In recent years, a production technology that utilizes the functions of living organisms such as microorganisms, which emit a small amount of carbon dioxide and have minimal environmental impact, so-called “bio-manufacturing” has been attracting attention.

Microorganisms inhabit various accommodates including soil, but among microorganisms present in those environments (so-called environmental microorganisms), only 1% of the microorganisms can be cultured, and most of the microorganisms present on the earth are unknown (Non Patent Literature 1). However, in recent years, with the advent of next generation sequencers, metagenomic analysis has become possible in which microbial genomes are collectively extracted from samples such as soil and sequencing is performed without culturing difficult-to-culture microorganisms that are challenging to propagate. Accordingly, it has become possible to prepare genetic information profiles of microorganisms that survive in various environments.

For example, when an environment in which unknown microorganisms are dominant is to be analyzed, draft genome information derived from unknown microorganisms is obtained through metagenome sequence assembly, and elucidation of the unknown functions is in progress. In fact, metagenomic analysis has been successively revealing new biological functions, such as unknown archaea (Non Patent Literature 2) dominant in biofilms within mine wastewater streams, uncultured phosphorus-accumulating bacteria (Non Patent Literature 3) that play a major role in phosphorus-removing waste water treatment systems, and unknown bacteria (Non Patent Literature 4) and archaea (Non Patent Literature 5) that are involved in the methane oxidation process in marine and freshwater environments.

However, in the generally performed metagenomic analysis method employing the shotgun method described in Patent Literature 1 and the like, the obtained gene information consists of a mixture of various microbial genome sequences, only slight overlaps in sequences are connected by a computer, and thus the accuracy of sequencing is low and the number of genes that can be obtained is not satisfactory.

### CITATION LIST

### Patent Literature

Patent Literature 1: WO 2016/079731

### Non Patent Literature

Non Patent Literature 1: Roger S Lasken, Curr Opin Microbiol, 10 (5): 510-516 (2007).
Non Patent Literature 2: Brett J Baker et al., Proc Natl Acad Sci US.A., 107 (19): 8806-8811 (2010).
Non Patent Literature 3: Hector Garcia Martin et al., Nat Biotechnol, 24 (10); 1263-1269 (2006).
Non Patent Literature 4; Katharina F Ettwig et al., Nature, 464 (7288): 543-548 (2010).
Non Patent Literature 5: Steven J Hallam et al., Science, 305 (5689): 1457-1462 (2004).

### SUMMARY OF INVENTION

### Technical Problem

As a result of intensive studies, the present inventors have developed a method in which one or several cells or cell-like structures are confined within a small section from a population of various cells or cell-like structures (in particularly, a microbial population) contained in a given sample, and then subjected to a step of lysing the cells or cell-like structures, followed by a step of amplifying the polynucleotides (nucleic acids such as genomic DNA, RNA) eluted in the small section. A plurality of small sections (for example, 5 to 100) are accommodated within a large section, and by subjecting the plurality of large sections simultaneously to the step of preparing a sequencing DNA library in parallel, a metagenomic library of the sample is obtained. The inventors have found that by subjecting this to a step of sequencing using a next generation sequencer As used herein, nucleic acid molecules corresponding to genes of novel organisms could be obtained with high efficiency, thereby completing the present disclosure.

That is, the present disclosure includes the following.

[Item 1] A nucleic acid library containing one or more nucleic acid molecules in one small section and a large section containing one or a plurality of the small sections.

[Item 2] The nucleic acid library according to the item, in which the large section contains two or more small sections.

[Item 3] The nucleic acid library according to any one of the items, in which the nucleic acid molecules contained in one small section include one or more nucleic acid molecules derived from a cell or cell-like structure and/or an amplification product obtained by amplification using the nucleic acid molecule as a template.

[Item 4] The nucleic acid library according to any one of the items, in which the nucleic acid library includes a library of metagenomes.

[Item 5] The nucleic acid library according to any one of the items, in which the nucleic acid or amplification product includes genomic DNA derived from a cell or a cell-like structure and/or an amplification product obtained by amplification using the genomic DNA as a template.

[Item 6] The nucleic acid library according to any one of the items, in which the large section contains 5 to 15 the small sections.

[Item 7] The nucleic acid library according to any one of the items, in which the one or more cells or cell-like structures include two or more types of cells or cell-like structures.

[Item 8] The nucleic acid library according to any one of the items, in which the small section is a gelled droplet, a droplet, or a coated droplet.

[Item 9] The nucleic acid library according to any one of the items, in which the gelled droplets are gelled with any gelling material selected from the group consisting of agarose, acrylamide, photocurable resin, polyethylene glycol, gelatin, sodium alginate, Matrigel, collagen, and hydrogel.

[Item 10] The nucleic acid library according to any one of the items, in which, in the amplification, the amplification is performed while maintaining a gel state within a gel capsule.

[Item 11] The nucleic acid library according to any one of the items, in which the gelled droplet has a diameter of about 1 to 250 μm.

[Item 12] The nucleic acid library according to any one of the items, in which the large section is a well forming portion of a microplate or a microtube.

[Item 13] A combination of nucleic acid libraries, comprising a plurality of nucleic acid libraries according to any one of the items.

[Item 14] The nucleic acid library or the combination of nucleic acid libraries according to any one of the items, in which the nucleic acids contained in the library include a barcode sequence unique to the large section.

[Item 15] The nucleic acid library or the combination of nucleic acid libraries according to any one of the items, in which the nucleic acids contained in the library contained in the small section include nucleic acids amplified to a predetermined length or more.

[Item 16] The nucleic acid library or the combination of nucleic acid libraries according to any one of the items, in which, in the library contained in the small section, a proportion of coding regions on contigs obtained from read sequences of the nucleic acids that are 1,000 bp or more is 5% or more.

[Item 17] The nucleic acid library or the combination of nucleic acid libraries according to any one of the items, in which, in the library contained in the small section, a full-length gene sequence content percentage in the protein-coding part (coding regions) on the contigs obtained from the read sequences of the nucleic acids is 20% or more.

[Item 18] The nucleic acid library or the combination of nucleic acid libraries according to any one of the items, in which, in the library contained in the small section, a full-length gene sequence content percentage in the protein-coding part (coding regions) on the contigs obtained from the read sequences of the nucleic acids is 30% or more.

[Item 19] A method for preparing a nucleic acid library, the method including:
a step of providing two or more cells or cell-like structures;
a step of generating a small section containing one or more cells or cell-like structures, from the cells or cell-like structures;
a step of lysing the cells or cell-like structures contained in the small section such that the nucleic acids in the cells or cell-like structures are eluted and retained within the small section;
a step of bringing the nucleic acids into contact with a nucleic acid amplification reagent to amplify the nucleic acids within the small section and obtain an amplification product, as necessary; and
a step of accommodating one or a plurality of the large sections containing the small sections containing the amplification product, in a container for nucleotide sequencing, as necessary.

[Item 20] The method according to the item, in which the large sections are all accommodated in the container.

[Item 21] The method according to any one of the items, in which the cells or cell-like structures are provided in a form of a suspension.

[Item 22] The method according to any one of the items, in which during the amplification, after cells or cell-like structures contained in the small section are lysed, nucleic acids containing the genome in the cells or cell-like structures are eluted into the small section and retained in the small section.

[Item 23] The method according to any one of the items, in which the step of obtaining the amplification product includes adding a barcode sequence unique to the large section to the nucleic acid.

[Item 24] The method according to any one of the items, in which the step of accommodating the small sections in the large sections includes removing nucleic acids other than nucleic acids amplified to a predetermined length or more.

[Item 25] The method according to any one of the items, in which the sample containing two or more cells or cell-like structures is a single sample.

[Item 26] The method according to any one of the items, in which the sample containing two or more cells or cell-like structures include one or more selected from the group consisting of soil (including seabed soil), seawater, river water, lake water, feces, saliva, skin, sputum, sludge (including activated sludge), industrial wastewater, tissues derived from animals and plants, and surgical irrigation solution.

[Item 27] The method according to any one of the items, in which the number of small sections accommodated in one large section is 2 or more.

[Item 28] The method according to any one of the items, in which the number of small sections accommodated in one large section is 5 to 15.

[Item 29] The method according to any one of the items, in which the cells or cell-like structures include two or more types.

[Item 30] A method for preparing a metagenomic library according to any one of the items, in which one or more or one or more types are 2 to 3 or 2 to 3 types.

[Item 31] The method according to any one of the items, in which the small section is a gelled droplet, a droplet, or a coated droplet.

[Item 32] The method according to any one of the items, in which the gelled droplets are gelled with any gelling material selected from the group consisting of agarose, acrylamide, photocurable resin, polyethylene glycol, gelatin, sodium alginate, Matrigel, collagen, and hydrogel.

[Item 33] The method according to any one of the items, in which the gelled droplet has a diameter of about 1 to 250 μm.

[Item 34] The method according to any one of the items, in which the step of generating the small sections is performed by previously containing a component having a gelling ability in the cell or cell-like structure suspension, then flowing the cell or cell-like Structure suspension having a gelling ability into a microchannel, and shearing the suspension with oil.

[Item 35] The method according to any one of the items, in which the large section is a well of a microplate or a microtube.

[Item 36] A method for analyzing nucleic acid sequences in cells or cell-like structures, the method including:
a step of providing two or more cells or cell-like structures;
a step of generating a small section containing one or more cells or cell-like structures, from the cells or cell-like structures;
a step of lysing the cells or the cell-like structures contained in the small section such that the nucleic acids in the cells or the cell-like structures are eluted and retained in the small section;
a step of obtaining an amplification product by bringing the nucleic acid into contact with the nucleic acid amplification reagent and amplifying the nucleic acid in the small section;
a step of accommodating one or a plurality of the large sections including the small sections containing the amplification product, in a container for nucleotide sequencing, as necessary;
a step of determining a nucleotide sequence of the amplification product;
a step of analyzing the nucleotide sequence obtained in the above step;
a step of analyzing the amino acid sequence encoded by the nucleotide sequence, as necessary; and
a step of analyzing a region encoding a gene, as necessary.

[Item 37] The method according to any one of the items, in which the large sections are all accommodated in the container.

[Item 38] The analysis method according to any one of the items, in which the step of obtaining the amplification product includes adding a barcode sequence unique to the large section to the nucleic acid.

[Item 39] The analysis method according to any one of the items, including combining small sections containing the amplification product into a smaller number of sections.

[Item 40] The analysis method according to any one of the items, including combining small sections containing the amplification product into one section.

[Item 41] The analysis method according to any one of the items, further including sorting only nucleic acids amplified to a predetermined length or more for the amplification product.

[Item 42] The analysis method according to any one of the items, in which the step of analyzing the region encoding the genes includes calculating a full-length gene sequence percentage.

[Item 43] A method for analyzing a metagenome, including:
a step of providing two or more cells or cell-like structures that constitute the metagenome;
a step of generating a small section containing one or more cells or cell-like structures, from the cells or cell-like structures;
a step of lysing the cells or the cell-like structures contained in the small section such that the nucleic acids in the cells or the cell-like structures are eluted and retained in the small section;
a step of obtaining an amplification product by bringing the nucleic acid into contact with the nucleic acid amplification reagent and amplifying the nucleic acid in the small section;
a step of accommodating one or a plurality of the large sections including the small section containing the amplification product, in a container for nucleotide sequencing, as necessary;
a step of determining a nucleotide sequence of the amplification product in the small section;
a step of analyzing the nucleotide sequence obtained in the above step to obtain nucleic acid sequence information of the metagenome;
a step of analyzing the amino acid sequence encoded by the nucleotide sequence, as necessary; and
a step of analyzing a region encoding a gene, as necessary.

[Item 43A] The method according to item 43, further including the features according to any one or a plurality of items 1 to 42.

[Item 44] An analysis system for a nucleic acid sequence in cells or cell-like structures, including:
a small section generation unit that generates a small section containing one or more cells or cell-like structures, from the cells or cell-like structures;
a nucleic acid lysis unit that lyses the cells or the cell-like structures contained in the small section such that the nucleic acids in the cells or the cell-like structures are eluted and retained in the small section;
an amplification reagent accommodation unit that accommodates a nucleic acid amplification reagent used for obtaining an amplification product by coming into contact with the nucleic acid and amplifying the nucleic acid in the small section;
an accommodation unit that accommodates one or a plurality of the large sections including the small sections containing the amplification product, as necessary;
a nucleotide sequencing unit that determines a nucleotide sequence of an amplification product in the small section;
a nucleotide sequence analysis unit that analyzes the nucleotide sequence obtained in the above step;
an amino acid sequence analysis unit that analyzes an amino acid sequence encoded by the nucleotide sequence, as necessary; and
a coding region analysis unit that analyzes a region encoding a gene, as necessary.

[Item 44A] The method according to item 44, further including the features according to any one or a plurality of items 1 to 43.

[Item 45] The method according to any one of items or the analysis system according to any one of items, in which the nucleotide sequences of the amplification products are determined simultaneously in parallel.

[Item 46] The method according to any one of items, or the analysis system according to any one of items, in which one or a plural number is 96 or more.

[Item 47] The method according to any one of the items, or the analysis system according to any one of the items, in which the determination of the nucleotide sequence is performed using a next generation sequencer.

[Item 48] A method for obtaining a nucleic acid sequence encoding genes in cells or cell-like structures and/or an amino acid sequence encoded by the nucleic acid sequence, the method including:
a step of providing two or more cells or cell-like structures;
a step of generating a small section containing one or more cells or cell-like structures, from the cells or cell-like structures;
a step of lysing the cells or cell-like structures contained in the small section such that the nucleic acids in the cells or cell-like structures are eluted and retained in the small section;
a step of bringing the nucleic acid into contact with a nucleic acid amplification reagent to amplify the nucleic acids in the small section to obtain an amplification product, as necessary;
a step of accommodating one or a plurality of the large sections including the small sections containing the amplification product, in a container for nucleotide sequencing, as necessary
a step of determining a nucleotide sequence of the amplification product;
a step of analyzing the nucleotide sequence obtained in the above step; and
a step of analyzing a region encoding genes in the nucleotide sequence to obtain a nucleic acid sequence encoding genes and/or an amino acid sequence encoded by the nucleic acid sequence.

[Item 48A] The method according to item 48, further including the features according to any one or a plurality of items 1 to 47.

[Item 49] A method for creating a database of sequences encoding genes in cells or cell-like structures, the method including:
a step of providing two or more cells or cell-like structures;
a step of generating a small section containing one or more cells or cell-like structures, from the cells or cell-like structures;
a step of lysing the cells or cell-like structures contained in the small section such that the nucleic acids in the cells or cell-like structures are eluted and retained in the small section;
a step of bringing the nucleic acid into contact with a nucleic acid amplification reagent to amplify the nucleic acids in the small section to obtain an amplification product, as necessary;
a step of accommodating one or a plurality of the large sections including the small sections containing the amplification product, in a container for nucleotide sequencing, as necessary;
a step of determining a nucleotide sequence of the amplification product;
a step of analyzing the nucleotide sequence obtained in the above step;
a step of analyzing a region encoding genes in the nucleotide sequence to obtain a nucleic acid sequence encoding genes and/or an amino acid sequence encoded by the nucleic acid sequence; and
a step of creating a database of sequences encoding genes by using the nucleic acid sequence encoding genes and/or the amino acid sequence encoded by the nucleic acid sequence, and as necessary, information on cells or cell-like structures.

[Item 49A] The method according to item 49, further including the features according to any one or a plurality of items 1 to 48.

[Item 50] A method for generating a data structure that constitutes a sequence encoding genes in cells or cell-like structures, the method including:
a step of providing two or more cells or cell-like structures;
a step of generating a small section containing one or more cells or cell-like structures, from the cells or cell-like structures;
a step of lysing the cells or cell-like structures contained in the small section such that the nucleic acids in the cells or cell-like structures are eluted and retained in the small section;
a step of bringing the nucleic acid into contact with a nucleic acid amplification reagent to amplify the nucleic acids in the small section to obtain an amplification product, as necessary;
a step of accommodating one or a plurality of the large sections including the small sections containing the amplification product, in a container for nucleotide sequencing, as necessary;
a step of determining a nucleotide sequence of the amplification product;
a step of analyzing the nucleotide sequence obtained in the above step;
a step of analyzing a region encoding a gene in the nucleotide sequence to obtain a nucleic acid sequence encoding genes and/or an amino acid sequence encoded by the nucleic acid sequence; and
a step of generating a data structure of sequences encoding genes, which are defined by the nucleic acid sequence encoding genes and/or the amino acid sequence encoded by the nucleic acid sequence, and as necessary, information on cells or cell-like structures.

[Item 50A] The method according to item 50, further including the features according to any one or a plurality of items 1 to 49.

[Item 51] A database generated by the method according to any one of items.

[Item 52] The database according to any one of items, in which the database includes information indicating that the database is generated by the method according to any one of items.

[Item 53] The database according to any one of items, in which the database contains full-length gene sequence percentage as a field.

[Item 54] The database according to any one of any one of the items, in which the database has a full-length gene sequence percentage of 20% or more for the nucleic acid sequence encoding contained genes and/or the amino acid sequence.

[Item 55] A data structure generated by the method according to any one of the items.

[Item 56] The data structure according to any one of the items, in which the data structure includes information indicating that the data structure is generated by the method according to any one of the items.

[Item 57] The data structure according to any one of the items, in which the data structure contains full-length gene sequence percentage as a field.

[Item 58] The data structure according to any one of any one of the items, in which the data structure has a full-length gene sequence percentage of 20% or more for the nucleic acid sequence encoding contained genes and/or the amino acid sequence.

[Item 59] The database or data structure according to any one of any one of the items, in which the database or data structure includes fields related to a coding sequence, and the fields related to a coding sequence are linked to contigs used in creating a genome database or a metagenome database.

[Item 60] The database or data structure according to any one of the items, in which the fields related to the coding sequences include a field that identifies whether the sequence is a complete code.

[Item 61] A method for analyzing an amino acid or nucleic acid sequence, the method including a step of analyzing the target amino acid or nucleic acid sequence using the nucleic acid sequence encoding genes or the amino acid sequence obtained by the method according to any one of the items, the database generated by the method according to any one of the items, the data structure generated by the method according to any one of the items, the database according to any one of the items, or the data structure according to any one of the items.

[Item 62] The method for analyzing an amino acid or nucleic acid sequence according to any one of the items, in which the analyzing step includes binning contigs to generate a genomic or metagenome database.

[Item 63] A method for preserving nucleic acid molecules, the method including a step of further preserving a large section accommodating small sections as it is or after adding a substance inhibiting DNA degradation at room temperature or less, after the final step of the method for preparing a nucleic acid library according to any one of the items.

[Item 64] The method according to any one of the items, in which the nucleic acid molecules are from microorganisms.

[Item 65] The method according to any one of the items, in which a temperature which is equal to or lower than room temperature is 4 °C or lower.

[Item 66] The method according to any one of the items, in which a temperature which is equal to or lower than room temperature is −20 °C or lower.

[Item 67] The method according to any one of the items, in which a temperature which is equal to or lower than room temperature is −80 °C or lower.

The present disclosure also provides the following.

[1] A metagenomic library including a large section containing genomic DNA derived from one or more or one or more types of cells or cell-like structures and/or an amplification product obtained by whole genome amplification using the genomic DNA as a template in one small section, and including one or a plurality of small sections.

[2] The metagenomic library according to the [1], in which the number of small sections in the large section is 2 to 300;

[3] The metagenomic library according to the [1], in which the number of small sections in the large section is 2 to 50.

[4] The metagenomic library according to the [1], in which the number of small sections in the large section is 5 to 15.

[5] The metagenomic library according to the [1], in which the number of small sections in the large section is 8 to 12.

[6] The metagenomic library according to the [1], in which the number of small sections in the large section is 10.

[7] The metagenomic library according to any one of the [1] to [6], in which one or more or one or more types is one or one type.

[8] The metagenomic library according to any one of the [1] to [6], in which one or more or one or more types are 2 to 3 or 2 to 3 types.

[9] The metagenomic library according to any one of the [1] to [8], in which the small section is a gelled droplet, a droplet, or a coated droplet.

[10] The metagenomic library according to the [9], in which the gelled droplets are gelled with any gelling material selected from the group consisting of agarose, acrylamide, photocurable resin, polyethylene glycol, gelatin, sodium alginate, Matrigel, collagen, and hydrogel.

[11] The metagenomic library according to the [9] or [10], in which the gelled droplet has a diameter of about 1 to 250 μm.

[12] The metagenomic library according to any one of the [1] to [11], in which the large section is a well of a microplate or a microtube.

[13] A metagenomic library including a plurality of metagenomic libraries according to any one of the [1] to [12].

[14] The metagenomic library according to the [13], in which the plurality of metagenomic libraries are any one selected from the group consisting of 384, 768, 1,152, 1,536, 1,920, 2,304, 2,688, and 3,072.

[15] A method for preparing a metagenomic library, including:
a step of suspending a sample containing two or more cells or cell-like structures in a suspension, and separating a fraction containing the cells or cell-like structures and the other fraction to obtain cells or cell-like structures;
a step of suspending the obtained cells or cell-like structures in a suspension, a step of generating a small section containing one or more or one or more types of cells or cell-like structures, from the cells or cell-like structure suspension;
a step of lysing the cells or cell-like structures contained in the small section, in which the entire genome in the cells or the cell-like structures is eluted into the small section and retained in the small section;
a step of bringing the entire genome into contact with a whole genome amplification reagent and amplifying the entire genome in the small section; and
a step of accommodating one or a plurality of small sections containing a whole genome amplification product in a large section.

[16] The method for preparing a metagenomic library according to the [15], in which the sample containing two or more cells or cell-like structures is a single sample.

[17] The method for preparing a metagenomic library according to the or [16], in which the sample containing two or more cells or cell-like structures include one or more selected from the group consisting of soil (including seabed soil), seawater, river water, lake water, feces, saliva, skin, sputum, sludge (including activated sludge), industrial wastewater, tissues derived from animals and plants, and surgical irrigation solution.

[18] The method for preparing a metagenomic library according to any one of the [15] to [17], in which the number of small sections to be accommodated in one large section is 2 to 300.

[19] The method for preparing a metagenomic library according to any one of the [15] to [17], in which the number of small sections to be accommodated in one large section is 2 to 50.

[20] The method for preparing a metagenomic library according to any one of the [15] to [17], in which the number of small sections to be accommodated in one large section is 5 to 15.

[21] The method for preparing a metagenomic library according to any one of the [15] to [17], in which the number of small sections to be accommodated in one large section is 8 to 12.

[22] The method for preparing a metagenomic library according to any one of [15] to [17], in which the number of small sections to be accommodated in one large section is 10.

[23] The method for preparing a metagenomic library according to any one of the [15] to [22], in which one or more or one or more types is one or one type.

[24] The method for preparing a metagenomic library according to any one of the [15] to [22], in which one or more or one or more types are 2 to 3 or 2 to 3 types.

[25] The method for preparing a metagenomic library according to any one of the [15] to [24], in which the small section is a gelled droplet, a droplet, or a coated droplet.

[26] The method for preparing a metagenomic library according to the [25], in which the gelled droplets are gelled with any gelling material selected from the group consisting of agarose, acrylamide, photocurable resin, polyethylene glycol, gelatin, sodium alginate, Matrigel, collagen, and hydrogel.

[27] The method for preparing a metagenomic library according to the or [26], in which the gelled droplet has a diameter of about 1 to 250 μm.

[28] The method for preparing a metagenomic library according to any one of [15] to [27], in which the step of generating the small sections containing one or more or one or more types of cells or cell-like structures, from the cells or cell-like structures, is performed by previously containing a component having a gelling ability in the cell or cell-like structure suspension, then flowing the cell or cell-like structure suspension having a gelling ability into a microchannel, and shearing the suspension with oil.

[29] The method for preparing a metagenomic library according to any one of the [15] to [28], in which the large section is a well of a microplate or a microtube.

[30] A highly efficient method for obtaining a microbial gene sequence, in which, after the final step of the method for preparing a metagenomic library according to any one of [15] to [29], the following steps are further performed;
a step of grouping a plurality of small sections in a large section into one;
a step of simultaneously and in parallel determining nucleotide sequences of amplification products in small sections grouped into one in a large section; and
a step of analyzing the nucleotide sequence obtained in the above step using a computer and estimating a region coding for a protein to obtain a gene.

[31] The highly efficient method for obtaining a microbial gene sequence according to the [30], in which the plurality of microbial gene sequences are any one selected from the group consisting of 384, 768, 1,152, 1,536, 1,920, 2,304, 2,688, and 3,072.

[32] The highly efficient method for obtaining a microbial gene sequence according to the [30] or [31], in which the determination of the nucleotide sequence is performed using a next generation sequencer.

[33] A method for preserving microbial genomic DNA, including a step of further preserving a large section accommodating small sections as it is or after adding a substance inhibiting DNA degradation at room temperature or less, after the final step of the method for preparing a metagenomic library according to any one of the to [29].

[34] The method for preserving microbial genomic DNA according to [33], in which a temperature which is equal to or lower than room temperature is 4 °C or lower.

[32] The method for preserving microbial genomic DNA according to [33], in which a temperature which is equal to or lower than room temperature is −20 °C or lower.

[36] The method for preserving microbial genomic DNA according to [33], in which a temperature which is equal to or lower than room temperature is −80 °C or lower.

In the present disclosure, it is intended that the one or more features may be provided in further combination in addition to the specified combination. Still further embodiments and advantages of the present disclosure will be appreciated by those skilled in the art upon reading and understanding the following detailed description as necessary.

As described above, the present disclosure provides a novel and highly efficient method for obtaining a gene sequence of organisms such as a microorganisms, nucleic acid (for example, metagenomes) libraries newly created for achieving the method, and the like. In one embodiment, there is provided a highly efficient method for obtaining a microbial gene sequence, the method including, for example, a step of obtaining cells or cell-like structures from a single sample; a step of suspending the obtained cells or cell-like structures in a suspension as necessary, a step of generating a small section containing one or more or one or more types of cells or cell-like structures, from the cell or cell-like structure suspension; a step of lysing the cells or cell-like structures contained in the small section and eluting nucleic acids such as genomic DNA into the small section; a step of performing a nucleic acid amplification reaction (for example, a whole genome amplification reaction) using the nucleic acid such as genomic DNA as a template; a step of accommodating small sections containing nucleic acid amplified (for example, whole genome amplification) products in a large section; and a step of simultaneously and in parallel determining the nucleotide sequences of whole genome amplification products in the large section.

The present disclosure solves the drawbacks observed when a method has been taken in which when obtaining microbial gene sequences from a soil sample containing microorganisms using a conventional shotgun metagenomic method, one metagenomic library is prepared from a single soil sample, the nucleotide sequence is determined, and the gene sequence is obtained (FIG. 1(A)). That is, the present disclosure makes it possible to efficiently obtain a large number of functional structural genes, which are usually difficult to reconstruct into the original gene sequence because the original gene sequence is reconstructed using the overlapping part of the obtained nucleotide sequence as a clue, but due to its enormous diversity, partial overlapping of sequences derived from different microorganisms, and the like in many cases. That is, in the highly efficient method for obtaining a microbial gene sequence of the present disclosure, small sections containing microbial cells are created from a single soil sample, genomic DNA derived from the microbial cells are eluted into the small sections, and then whole genome amplification reaction is caused to occur in these small sections, 10 small sections containing DNA amplification fragments are accommodated in, for example, large sections, and 384 large sections are prepared, for example, to prepare 384 metagenomic libraries (FIG. 2). As a result, even when various microbial cells are present in the sample, detailed analysis can be performed. Consequently, compared to conventional methods, the gene acquisition efficiency from a single soil sample increases dramatically, achieving the efficient acquisition of a large number of functional structural genes.

The present disclosure provides a novel highly efficient method for obtaining a microbial gene sequence. Currently, humanity faces a wide range of environmental challenges, one of which is the conservation of biodiversity. On the earth, various organisms inhabit diverse environments and coexist while directly or indirectly supporting each other. Humanity coexists with other living organisms as a member of the ecosystem and is supported by various benefits (ecosystem services) provided by biodiversity, including diverse ecosystems such as forests, rivers, and oceans, diverse biological species of plants, animals, and microorganisms, and diverse genotypes within each species.

Specifically, human life is established by benefits such as supply of oxygen, purification of water, supply of raw materials such as food, fiber, and wood, supply of medicinal resources, and mitigation of damage due to a storm, a flood, and the like, in addition to benefits related to existence foundation of life such as rich soil. However, due to the expansion of human economic activities, the deterioration of the habitat environment of organisms and the destruction of ecosystems have progressed, and the extinction of biological species has progressed rapidly, and the biological diversity constituting the source of the ecosystem services is threatened. Amid these circumstances, the importance of halting and restoring biodiversity loss has come to be recognized by the international community. Biodiversity can also be regarded as genetic diversity. Genetic diversity is also directly linked to the adaptability of an entire species inhabiting a given environment. When genetic diversity is rich and many different species of the same organism exist, then even when the environment changes or unforeseen disturbances occurs, individuals with unique traits (genetic characteristics) that can adapt to this can increase their chances of surviving and avoiding extinction. In such a point, the present disclosure provides a technology that makes it possible to ensure genetic diversity and efficiently ensure and store various genetic resources.

### Advantageous Effects of Invention

The metagenomic library and the highly efficient method for obtaining a microbial gene sequence of the present disclosure are useful for highly efficiently obtaining novel genes from samples containing microbial cells such as soil.

### BRIEF DESCRIPTION OF DRAWINGS

FIG. 1 is a schematic diagram comparing a metagenomic library preparation process of the present disclosure with a conventional shotgun metagenomic method and a mini-metagenomic method.

FIG. 2 is a diagram showing ultra-parallel processing steps from preparation of small sections containing microbial cells to preparation of a metagenomic library of the present disclosure composed of large sections containing a plurality of small sections filled with genomic DNA amplification products, up to the point where the metagenomic library is subjected to sequencing by a next generation sequencer.

FIG. 3 is a diagram showing a step of obtaining a small section containing amplification fragments derived from microbial cell genomic DNA using each of a gelled droplet (a), a droplet (b), and a coated droplet (c) as the small sections.

FIG. 4 is a diagram showing a morphology in which a monococcus undergoes division, and a type of coccus possessing an identical genome exists in nature in a chained form with a plurality of cells.

FIG. 5(a) is an image diagram in the case of preparing droplets containing one microbial cell per droplet, and FIG. 5(b) is an image diagram in the case of preparing droplets containing two microbial cells per droplet.

FIG. 6 is an image diagram of a metagenomic library of the present disclosure prepared using a microplate.

FIG. 7 is an image diagram of a metagenomic library of the present disclosure prepared using a microtube.

FIG. 8 is a diagram showing a distribution of gene lengths of gene groups obtained using a shotgun metagenomic sequencing method and the method according to the present disclosure,

FIG. 9 is a diagram comparing the number of non-redundant genes obtained when microbial genes are obtained from a soil sample by the method of the present disclosure and when microbial genes are obtained by the shotgun metagenomic sequencing method.

FIG. 10 is a diagram showing a relationship between the number of genes and the number of wells obtained when 10 gelled droplets per well are accommodated in a microplate.

FIG. 11 shows the results of an examination conducted in Example 6, indicating a percentage of genes with a length of 1,000 bp or less in a protein-coding part (coding region) on contigs obtained from nucleic acid read sequences. The X-axis indicates the gene length, and the Y-axis indicates the percentage with respect to the whole.

FIG. 12 shows a full-length gene sequence percentage (full-length gene sequence content percentage for a library) of the results of Examples of the present disclosure (in the protein-coding part (coding region) on contigs obtained from nucleic acid read sequences).

### DESCRIPTION OF EMBODIMENTS

Hereinafter, the present disclosure will be described while showing the best mode. It is to be understood that throughout the present specification, the singular forms of expression also include the concept of the plural forms thereof, unless otherwise stated. Thus, it is to be understood that the singular article (for example, in English, “a”, “an”; “the”, and the like are used) also includes the concept of the plural forms, unless otherwise stated. It is also to be understood that the terms used in the present disclosure are used in the sense commonly used in the field unless otherwise noted. Thus, unless defined otherwise, all technical and scientific terms used As used herein have the same meaning as commonly understood by those skilled in the art to which the present disclosure belongs. In case of conflict, the present specification (including definitions) will take precedence. As used As used herein, the term “step” includes not only steps that are independent of other steps but also those that may not be clearly distinguishable from other steps, as long as the objective of the step is achieved. Numerical ranges indicated using “to” as used As used herein include the numerical values described before and after “to” as the minimum value and the maximum value, respectively. In the numerical range described stepwise As used herein, the upper limit value or the lower limit value described in one numerical range may be replaced with the upper limit value or the lower limit value of the numerical range described in another stage. In addition, in the numerical range described in the present text, the upper limit value or the lower limit value of the numerical range may be replaced with a value shown in Examples.

### DEFINITIONS

Descriptions and definitions of terms and general techniques used in the present disclosure are set forth in conjunction with the description of the present disclosure.

As used herein, the terms “polynucleotide”, “oligonucleotide”, and “nucleic acid” are used interchangeably and refer to a polymer of nucleotides of any length. The term “nucleic acid” is also used As used herein interchangeably with genes, DNA (for example, cDNA), RNA (for example, mRNA), oligonucleotides, and polynucleotides. As used As used herein, the term “nucleotide” may be natural or non-natural. As used herein, the term “gene” refers to a factor that defines genetic traits, and the term “gene” may refer to a “polynucleotide”, “oligonucleotide”, and “nucleic acid”. When referring to a nucleic acid as an isolated molecule, that is, focusing on the individual aspects as a substance, the term “nucleic acid molecule” may be used.

As used herein, the term “metagenome” refers to the total sum of genomes of organisms (for example, may be microorganisms) constituting a community. Therefore, examples of metagenomes include the total genome obtained by extracting DNA or other genomes from cells or cell-like structures such as microorganisms contained in various samples, such as soil (including seabed soil), seawater, river water, lake water, feces, saliva, skin, sputum, sludge (including activated sludge), industrial wastewater, tissues derived from animals and plants, and surgical irrigation solution, as well as the constituent substances thereof (nucleic acids such as DNA and RNA). Here, for example, a fecal sample may contain the gut microbiota of the organism from which the fecal sample is derived, a saliva sample may contain the oral microbiota of the organism from which the saliva sample is derived, and a skin sample may contain the skin microbiota of the organism from which the skin sample is derived, making these suitable samples for obtaining a metagenome. As used herein, the term “metagenomic analysis” refers to comprehensively determining the sequence of DNA extracted from a sample (community).

Note that, as a previous step of the genome extraction, a so-called culture step can be included in which cells such as microorganisms in the sample are accommodated under conditions that can divide. Here, the term “cell” refers to a particle containing a molecule having genetic information, and refers to any particle capable of self-replication. Examples of the cell include bacterial cells, fungal cells, cells of unicellular organisms, and cells derived from multicellular organisms. The term “cell-like structure” refers to any particle containing a molecule having genetic information. Examples of the cell-like structure include intracellular organelles such as mitochondria, cell nuclei, and chloroplasts; extracellular vesicles; and viruses. In addition, in the 2000s, with the development of next generation sequencing technology, it became clear that environments such as those described above is filled with microorganisms exhibiting a level of diversity far beyond human imagination, and that an astonishing amount of uncultured microorganisms with unknown functions exist there, forming the foundation of the biological ecosystem. Therefore, such an environment can be a source of obtaining a novel gene having a novel function that is industrially applicable in the present disclosure.

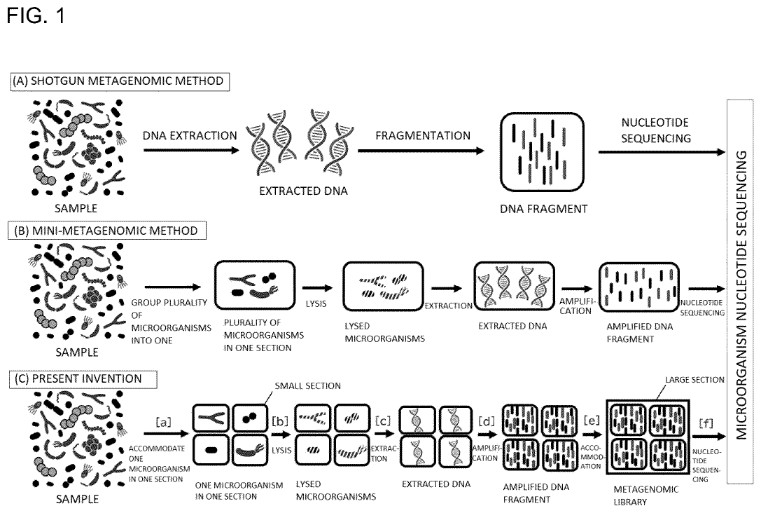

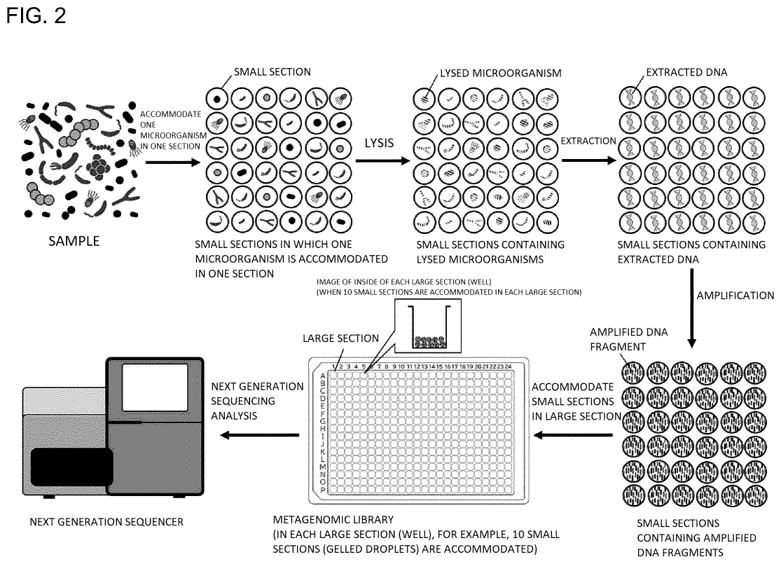

As used herein, the term “library” of “nucleic acid” refers to a collection of a plurality of nucleic acids, and when the target is a metagenome, the term “library” refers to a “metagenomic library.” In an embodiment of the present disclosure, the metagenomic library is a genomic library constructed from genomic DNAs of various cells or cell-like structures present in a sample. More specifically, as shown in FIGS. 1(C) and 2, the metagenomic library refers to both a metagenomic library including a large section that includes one or a plurality of small sections containing amplified DNA fragments obtained by separating cells or cell-like structures such as microorganisms from a single sample containing cells or cell-like structures such as microorganisms, accommodating one or more or one or more types of cells or cell-like structures per small section, lysing the cell partition walls of the cells or cell-like structures, removing contaminants other than genomic DNA, and amplifying the genomic DNA; and a metagenomic library including a collection containing a plurality of such large sections. Without intending to be limiting, the metagenomic library of the present disclosure has a feature that it is possible to obtain gene sequences derived from cells or cell-like structures such as microorganisms with higher efficiency, unlike conventional ones. In certain embodiments of the present disclosure, as used As used herein, the term “gene” refers to a structural gene that defines the primary structure of a protein.

As used herein, the term “small section” refers to a chamber enclosed by a barrier that may have a function of allowing specific molecules to pass while preventing the passage of other molecules. The barrier used for the small section is typically one (for example, a semipermeable membrane) that allows the passage of reagents necessary for nucleic acid amplification while preventing the passage of the nucleic acid amplification target. Therefore, typically, regarding the exchange of substances across the barrier, only the reagents necessary for nucleic acid amplification can pass through, ensuring that the nucleic acid target remains retained within the small section under normal conditions. Meanwhile, the reagents necessary for nucleic acid amplification (for example, polymerase, buffer components, primers (nucleic acid polymers with a molecular weight below a certain level), and components necessary for the exertion of polymerase enzymatic activity) can be externally added. Therefore, in this aspect, the small section of the present disclosure can efficiently amplify nucleic acids derived from contained cells or cell-like structures. In a preferred embodiment, the small sections of the present disclosure are advantageous structures that are not substantially destructed by means (agents) or conditions (heating, shearing, and the like) that lyse the cells or cell-like structures. In still another preferred embodiment, the small section can advantageously provide conditions under which the nucleic acid molecule can be homogeneously amplified when the target is a nucleic acid molecule. While not wishing to be bound by theory, it is possible to uniformly amplify a target nucleic acid molecule to be amplified (typically, a genomic nucleic acid molecule) under a condition that allows homogeneous amplification, and to comprehensively obtain substantially all sequence information of the target nucleic acid. In this case, even when there are a plurality of types (for example, about 10 types) of amplified nucleic acid sequences (for example, genome sequences), the types can be distinguished from each other, and thus sequence information such as genome information can be restored with high accuracy. The small sections can have a minute volume of nano-picoliter per section, and can be used as a minute reaction field for individually processing or individually analyzing cells or cell-like structures such as microorganisms. For example, the small sections of tens of thousands to millions can be created from a reaction liquid at a microliter level. Therefore, as shown in FIG. 2, there is a feature that ultra-parallel reactions can be executed. In the present disclosure, the term “ultra-parallel” refers to a broad concept intended to process a sample, a cell, genomic DNA, an amplified DNA fragment, and a large number of small sections and/or a large number of large sections containing the same in parallel for the purpose of rapidly and efficiently obtaining a large number of genes from a target sample. Examples of the “small section” include, but are not limited to, gelled droplets, droplets, and coated droplets.

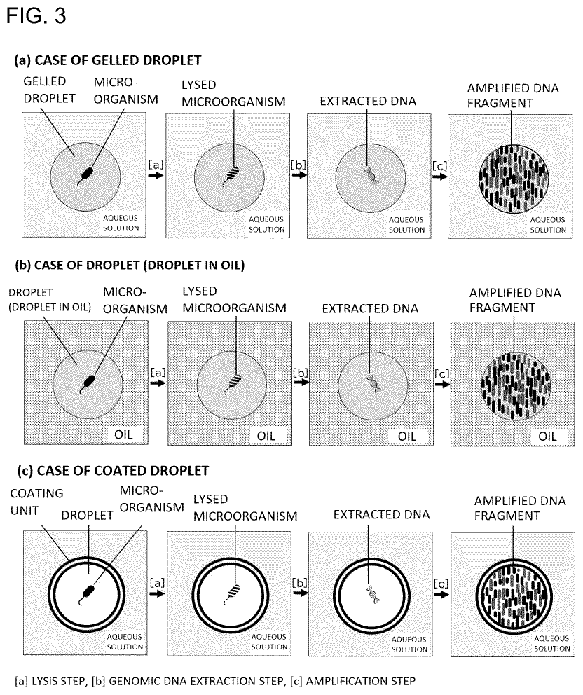

As used herein, the term “gelled droplet” refers to a gel-like particulate structure capable of retaining cells or cell-like structures therein, as shown at the left end of FIG. 3(a). Here, the term “gel” refers to a state where a polymer substance or colloidal particles in a colloidal solution form a network structure as a whole by their interaction, and have lost fluidity while containing a large amount of a liquid phase as a solvent or a dispersion medium. Examples of the gelling material include agarose, acrylamide, a photocurable resin, polyethylene glycol, gelatin, sodium alginate, Matrigel, collagen, and hydrogel. The term “droplet” refers to a collection of liquid particles with surface tension capable of retaining cells or cell-like structures therein, for example, water droplets in oil (water in oil (W/O)), oil droplets in water (oil in water (O/W)), but from the viewpoint of DNA amplification reaction efficiency, water droplets in oil (water in oil (W/O)) is preferable. An image of a water droplet in oil is shown at the left end of FIG. 3(b). The term “coated droplet” refers to a droplet coated with a gel-like substance capable of retaining cells or cell-like structures therein, as shown at the left end of FIG. 3(c). In the coated droplet, the droplet itself may be an aqueous droplet such as an aqueous solution or a sol-like droplet. Here, the “sol” refers to a colloid having fluidity and containing a liquid as a dispersion medium. The droplet and the coated droplet can be prepared by a method known in the relevant technical field. As used herein, the term “hydrogel” refers to a substance in which a solvent or a dispersion medium retained by a network structure of polymer substances or colloidal particles is water.

As used herein, the term “large section” refers to a chamber enclosed by a barrier that can accommodate one or a plurality of the small sections. For example, examples of the “large section” include a well forming portion (also simply referred to as a “well” unless otherwise noted As used herein) of a microplate, and a microtube, but are not limited thereto as long as one or a plurality of small sections can be accommodated. The microplate has various numbers of wells, for example, 6 wells, 12 wells, 24 wells, 96 wells, 384 wells, 1,536 wells, and 6,144 wells, and a microplate having a suitable number of wells can be used according to a purpose. Usually, for example, when the metagenomic library of the present disclosure is prepared from a soil sample, as a specific example, a 384-well microplate can be suitably used, and by using a plurality of 384-well microplates in parallel at the same time, in the case of two microplates, 768 wells can be processed; in the case of three microplates, 1,152 wells; in the case of four microplates, 1,536 wells; in the case of live microplates, 1,920 wells; in the case of six microplates, 2,304 wells; in the case of seven microplates, 2,688 wells; and in the case of eight microplates, 3,072 wells can be processed at once. The number of plates can be further increased as necessary, thereby increasing the number of wells (number of large sections). As used herein, the “large section” and the “small section” can also be simply expressed as a “section” and a “subsection”.

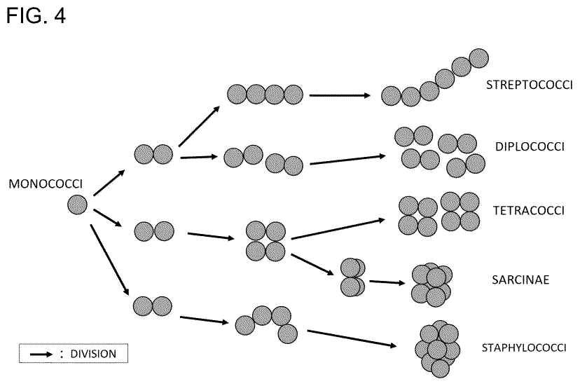

As used herein, the term “single cell” means one or more or one or more types of cells or cell-like structures. Here, as shown in FIG. 4, the term “one type of cell” refers to bacteria that contain the same genomic DNA and are made up of a plurality of cells that exist in nature in the form of diplococci, tetracocci, sarcinae, streptococci, staphylococci, and the like, which are formed by division of monococci, which originally consisted of a single cell. Similarly, two or two types of cells or cell-like structures refer to as “double cells”, and three or three types of cells or cell-like structures are referred to as “triple cells”. In the step of accommodating a desired number of cells or cell-like structures such as microorganisms in each of the sections described below, the metagenomic library of the present disclosure contains 1 to 3 or 1 to 3 types of cells or cell-like structures or 2 to 3 or 2 to 3 types of cells or cell-like structures in one section, more preferably 1 to 3 or 1 to 3 types of cells or cell-like structures in one section, and most preferably 1 or 1 type of cell of cell-like structure in one section. In particular, from the viewpoint of obtaining a high-quality gene group, it is most preferable to accommodate one or one type of cell or cell-like structure in one section.

As used herein, the term “single cell level” refers to processing in a state where genetic information contained in one or one type of cell or cell-like structure is distinguished from genetic information contained in other cells or cell-like structures. For example, in the case of amplifying a polynucleotide such as genomic DNA at the “single cell level”, each amplification is performed in a state where a polynucleotide in a certain cell and a polynucleotide in another cell can be distinguished. The term “double cell level” means that the processing is performed in a state where two or two types of cells or cell-like structures are contained, and similarly, the “triple cell level” means that the processing is performed in a state where three or three types of cells or cell-like structures are contained. For the purpose of obtaining more gene groups from the same sample, treatment may be performed at the double cell level or the triple cell level or higher (for example, 10 or 10 types of cells or cell-like structures).

As used herein, the terms “amplification”, “amplify”, “amplifying”, or “amplification reactions” and their derivatives herein generally refer to the action or process by which at least a part of a nucleic acid molecule (referred to as a template nucleic acid molecule) is replicated or copied into at least one additional nucleic acid molecule. The additional nucleic acid molecule optionally includes a sequence that is substantially identical to or substantially complementary to at least some part of the template nucleic acid molecule. The template target nucleic acid molecule can be single stranded or double stranded. The additionally obtained replicating nucleic acid molecules can be independently single stranded or double stranded. In some embodiments, amplification includes a template-dependent in vitro enzyme-catalyzed reaction for generation of at least one copy of at least some part of a target nucleic acid molecule or for generation of at least one copy of a target nucleic acid sequence that is complementary to at least some part of a target nucleic acid molecule. Amplification optionally includes linear or exponential replication of nucleic acid molecules. In some embodiments, such amplification is performed using isothermal conditions, and in other embodiments, such amplification can include thermocycling. In some embodiments, amplification is multiple amplification involving simultaneous amplification of a plurality of target sequences in a single amplification reaction. At least some target sequences can be positioned on the same or different target nucleic acid molecules included in a single amplification reaction. In some embodiments, “amplification”, whether alone or in combination, includes amplification of at least some part of a DNA-based nucleic acid and/or an RNA-based nucleic acid. The amplification reaction can include a single-stranded or double-stranded nucleic acid substrate and can further include any amplification process known to those skilled in the art. In some embodiments, the amplification reaction includes polymerase chain reaction (PCR). In some embodiments, the amplification reaction includes isothermal amplification. As used herein, it is preferable for amplification to occur while maintaining the gel state within small sections such as gel capsules or gelled droplets, as this can be advantageous.

As used herein, the term “whole genome amplification” means that when a nucleic acid to be amplified is a genome, the entire genome is amplified. The whole genome amplification can be performed, for example, by a multiple displacement amplification (MDA) method in which the entire genome is amplified through random priming using a DNA polymerase having a strand displacement activity, such as phi29 DNA polymerase or Bst DNA Polymerase.

As used herein, when referring to a collection of nucleic acid sequences or amino acid sequences, the “full-length gene sequence percentage” refers to the number obtained by dividing the number of samples (for example, a nucleic acid molecule or polypeptide molecule) encoding a complete structural gene by the number of all samples examined when the amino acid sequence encoded by the nucleic acid sequence is analyzed. Typically, a case where the full length of the coding region (CDS) of a gene is contained within a contig is labeled as “complete”, and a case where the full length of the coding region (CDS) of a gene is not contained within a contig is labeled as “incomplete”. “Complete/complete+incomplete” can be displayed as the “full-length gene sequence percentage”. When referring to a database or data structure of nucleic acid or amino acid sequences, when contigs obtained after joining nucleic acid read sequences obtained from a nucleic acid library are subjected to computer analysis to estimate a region that seems to encode a protein, the number of coding regions including a full-length sequence starting with a start codon and ending with a stop codon can be evaluated as a ratio obtained by dividing the number of such coding regions by the total number of coding regions obtained. Since databases, data structures, and the like have contigs as data, such a case is referred to as a full-length gene sequence percentage, and a nucleic acid library on which reads and read sequences are based is referred to as a “full-length gene sequence content percentage” as described below. Such a concept can be said to be one parameter for displaying quality in a sample such as a database and a data structure of nucleic acids such as genomes or metagenomes, and a nucleic acid library from which genomes or metagenomes are derived, and the database and the data structure may have a full-length gene sequence percentage of at least 10% or more, preferably 15% or more, 20% or more, 25% or more, 30% or more, 35% or more, 40% or more, 45% or more, or 50% or more. When actually calculated in the present disclosure, the determination can be made by examining the length (coding region) of the protein-coding part of the contig obtained (joined) from the read sequence of the nucleic acid. For example, it is necessary that both ATG and the termination codon are present, and these can be identified and calculated as sequences encoding genes (proteins) using well-known methods in the relevant field.

As used herein, or when referring to a collection of nucleic acids or polypeptides, such as a sample containing a nucleic acid sequence or amino acid sequence, a library, or the like, the “full-length gene sequence content percentage” refers to, when discussing a nucleic acid library such as the library of the present disclosure, a percentage obtained by dividing the number of coding regions that contain full-length gene sequences (which start with a start codon and end with a stop codon) by the total number of coding regions obtained, after computational analysis of contigs, which are generated by assembling the read sequences of nucleic acids contained in the library, and estimating the regions (coding regions) presumed to encode proteins. The full-length gene sequence content percentage of the library is useful as a value indicating the evaluation of the quality of the library. Such a concept can be said to be one parameter for displaying quality in a sample such as a nucleic acid library used for generating databases and data structures of nucleic acids such as genomes or metagenomes, and the library may have a full-length gene sequence content percentage of at least 10% or more, preferably 15% or more, 20% or more, 25% or more, 30% or more, 35% or more, 40% or more, 45% or more, or 50% or more. When actually calculated in the present disclosure, the determination can be made by examining the length (coding region) of the protein-coding part of the contig obtained (joined) from the read sequence of the nucleic acid. For example, it is necessary that both ATG and a termination codon are present, these can identify and calculate a sequence encoding a gene (protein) in a manner well known in the field, and when applied to a library, the obtained values can be displayed as the full-length gene sequence content percentage.

As used herein, the term “coding region” refers to a region corresponding to a coding region (CDS) considered to encode a gene in a contig obtained from read sequences of nucleic acid in a collection of nucleic acids, a library or the like. When the coding region is identified, the length is identified, and the coding region of which the length is, for example, a certain length (for example, 1,000 bp) or more can be used as an index of quality evaluation. As used herein, it is considered that the proportion of those having a length of typically 1,000 bp or more can be used as an index of a nucleic acid library. Then, the proportion obtained by dividing the number of coding regions having a length of 1,000 bp or more by the total number of coding regions can be used as an index of the quality and accuracy of a nucleic acid library in the present disclosure, and only about 3% could be achieved in the conventional technology. In the present disclosure, this can be achieved at a high proportion of 3% or more, preferably 5% or more, 10% or more, 15% or more, or 20% or more.

According to one embodiment, one aspect (FIG. 1(C)) in a case where the method for preparing a nucleic acid library of the present disclosure is directed to a metagenomic library is shown in FIG. 1 in comparison with a conventional method, a shotgun metagenomic method (FIG. 1(A)) and a mini-metagenomic method (FIG. 1(B)), but the method for preparing a metagenomic library of the present disclosure is not limited to this method (FIG. 1(C). In this exemplary embodiment, as shown in [a] to [e] of FIG. 1(C), the method for preparing a nucleic acid library such as metagenomes of the present disclosure can include a step of accommodating a desired number of cells or cell-like structures such as microorganisms in each small section ([a] of FIG. 1(C)), a step of lysing the cells or cell-like structures ([b] of FIG. 1(C)), a step of extracting nucleic acid molecules such as genomic DNA from the lysed cells or cell-like structures as necessary ([e] of FIG. 1(C)), a step of amplifying the nucleic acid molecules ([d] of FIG. 1(C)), and a step of accommodating one or a plurality of small sections containing the amplified nucleic acid molecules in a large section ([e] of FIG. 1).

As used herein, the term “microorganism” means at least one type of organism such as a virus, a bacterium, a fungus, a protist, a microalgae, or an animal or plant cell, but is not limited thereto. Even when plant or animal cells are individually large and not considered microorganisms, it is understood that when handled separately as individual cells, the plant or animal cells fall within the concept of microorganisms in the present disclosure. Examples of the bacteria include eubacteria and archaea. Examples include eubacteria including gram-positive bacteria belonging to the genera Staphylococcus, Streptococcus, Enterococcus, Micrococcus, Bacillus, and Leuconostoc, and gram-negative bacteria belonging to the genera Acetobacter, Gluconobacter, Gluconacetobacter, Pseudomonas, Acinetobacter, and Escherichia; and archaea belonging to the genera Acidianus, Metallosphaera, Stygiolobus, Sulfolobus, Sulfurococcus, Sulfurisphaera, Aeropyrum, Desulfurococcus, Seteria, Staphylothermus, Thermodiscus, Ignicoccus, Thermosphaera, Sulfophobococcus, Hyperthermus, Pyrodictium, Pyrolobus, Pyrobaculum, Thermoproteus, Thermofilum, Caldococcus, Archaeoglobus, Ferroglobus, Methanothermus, Methanobacterium, Methanothermobacter, Methanosphaera, Methanococcus, Methanothermococcus, Methanocaldococcus, Methanoignis, Methanosarcina, Pyrococcus, Thermococcus, Thermoplasma, and Picrophilus. In addition, it is possible to perform comprehensive analysis for each cell in a sample in which a plurality of these examples are mixed.

As used herein, examples of fungi include Ascomycota, Myxomycota, Phycomycetes, Basidiomycota, and Fungi Imperfecti. Examples of yeasts classified under Ascomycota include those belonging to the genera Saccharomyces, Kluyveromyces, Candida, Pichia, and Schizosaccharomyces.

### PREFERRED EMBODIMENTS

Although a description of preferred embodiments is set forth below, it is to be understood that this embodiment is illustrative of the present disclosure and that the scope of the present disclosure is not limited to such preferred embodiments. It should be understood that those skilled in the art can also easily make alterations, modifications, and the like within the scope of the present disclosure with reference to the following preferred examples. Therefore, it is apparent that those skilled in the art can appropriately make modifications within the scope of the present disclosure in view of the description used herein. It is also understood that the following embodiments of the present disclosure may be used alone or in combination.

### (Nucleic Acid Library and Method for Preparing the Same)

In one aspect, the present disclosure provides a nucleic acid library that contains one or more nucleic acid molecules in one small section and a large section containing one or a plurality of the small sections. In this aspect of the present disclosure, the barrier used for the small section to be used is typically one (for example, a semipermeable membrane) that allows the passage of reagents necessary for nucleic acid amplification while preventing the passage of the nucleic acid amplification target. Therefore, typically, regarding the exchange of substances across the barrier, only the reagents necessary for nucleic acid amplification can pass through, ensuring that the nucleic acid target remains retained within the small section under normal conditions. Meanwhile, the reagents necessary for nucleic acid amplification (for example, polymerase, buffer components, primers (nucleic acid polymers with a molecular weight below a certain level), and components necessary for the exertion of polymerase enzymatic activity) can be externally added. Therefore, in this aspect, the small section of the present disclosure can efficiently amplify nucleic acids derived from contained cells or cell-like structures.

In a preferred embodiment, the small sections of the present disclosure are advantageous structures that are not substantially destructed by means (agents) or conditions (heating, shearing, and the like) that lyse the cells or cell-like structures.

In still another preferred embodiment, the small section can advantageously provide conditions under which the nucleic acid molecule can be homogeneously amplified when the target is a nucleic acid molecule. While not wishing to be bound by theory, it is possible to uniformly amplify a target nucleic acid molecule to be amplified (typically, a genomic nucleic acid molecule) under a condition that allows homogeneous amplification, and to comprehensively obtain substantially all sequence information of the target nucleic acid. In this case, even when there are a plurality of types (for example, about 10 types) of amplified nucleic acid sequences (for example, genome sequences), the types can be distinguished from each other, and thus sequence information such as genome information can be restored with high accuracy, and there is an unprecedented utility value.

In a certain embodiment of the present disclosure, in that case, in order to determine from which large section the obtained nucleotide sequence is derived, each nucleic acid molecule may contain a sequence corresponding to a unique barcode sequence (also referred to as a nucleic acid barcode) specific to each large section. The length of the barcode sequence contained in the nucleic acid molecule may be 4 to 12 bases, preferably 6 to 10 bases, and most preferably 8 to 10 bases. Two or more types of nucleic acid barcodes may be used in combination, and according to the combination, for example, even when 8 plates having 384 large sections are simultaneously used in parallel (3,072 large sections in total), it is possible to distinguish each large section by using 3,072 types of nucleic acid barcodes having different sequences for each large section. When the number of types or combinations of barcode sequences to be prepared is further increased, ultra-parallel processing using still more large sections can be supported. Note that, when adding the nucleic acid barcode sequence for identifying the position of the large section, it is important to know which sequence of the nucleic acid barcode molecule was added to which position of the well, and such information can be separately stored and recorded.

In addition, in another preferred embodiment, the nucleic acids contained in the libraries contained in the small sections may be configured to include nucleic acids having substantially a predetermined length or more. Here, the predetermined length may be a minimum length in order to be comprehensive for analyzing the genome sequence. In addition, the term “substantially” means that the presence of nucleic acids having the predetermined length or more enables the analysis of nucleic acid sequences, such as genomes, and as long as the nucleic acid analysis is not inhibited, the presence of a small amount of shorter nucleic acids is acceptable. Containing nucleic acids having a predetermined length or more can be achieved by generating nucleic acids amplified to the predetermined length or more and selecting the nucleic acids. In order to obtain nucleic acids having a predetermined length or more, for example, in a case where the small sections are gelled droplets, the gelled droplets after nucleic acid amplification are centrifugally washed, for example, with phosphate buffered saline (PBS) as a washing solution, then stained with a DNA intercalator, for example, with a fluorescent dye (for example, SYBR green (5760A, Takara Bio Inc.), EvaGreen (31000, Cosmo Bio Co., Ltd.)), and observed with a flow cytometer, whereby the gelled droplets containing genomic DNA amplified to a predetermined amount or more are sorted, and a predetermined number of the gelled droplets (small sections) are accommodated in each well (large sections) of the microplate (for example, HSP3801, Bio-Rad Laboratories, Inc.).

In one embodiment, in the library of the present disclosure, the large section includes two or more of the small sections. Even when a plurality of small sections were included in the large sections, there was no unexpected trouble in the subsequent analysis of the nucleic acid sequence. On the other hand, in the present disclosure, due to the presence of the small sections, cells or cell-like structures individually present therein or nucleic acids derived therefrom can be individually amplified, and as necessary, can be amplified to include a barcode sequence.

In another embodiment, in the library of the present disclosure, the nucleic acid molecule contained in one small section includes a nucleic acid molecule derived from one or more cells or cell-like structures and/or an amplification product obtained by amplification using the nucleic acid molecule as a template. The number of cells or cell-like structures contained in one small section may be two or more, and in this case, the two or more cells or cell-like structures may be of the same species or of different species, and when there are n (n is a positive integer) cells or cell-like structures, there may be 2 to n types of cells or cell-like structures in the case of different types.

In certain embodiments, a nucleic acid library of the present disclosure includes a library of metagenomes.

In one embodiment, the nucleic acid or amplification product contained in the small section includes genomic DNA derived from a cell or a cell-like structure and/or an amplification product obtained by amplification using the genomic DNA as a template.

In one embodiment, the large section may advantageously include 2 or more small sections, preferably 3 or more, 4 or more, 5 or more, 10 or more, or 20 or more small sections, and usually 2 to 300 small sections, preferably 2 to 50, 5 to 15, 8 to 12, or about 10 small sections, but the present disclosure is not limited thereto.

In one embodiment, the one or more cells or cell-like structures from which the nucleic acid molecule is derived includes two or more types of cells or cell-like structures. The case of containing two or more types of cells or cell-like structures also includes the case of constituting a metagenome.

In one embodiment, the small section is advantageously a gelled droplet, a droplet or a coated droplet. While not wishing to be bound by theory, in any of the gelled droplet, the droplet, or the coated droplet, the passage of reagents necessary for nucleic acid amplification is allowed while preventing the passage of the nucleic acid amplification target. Therefore, typically, regarding the exchange of substances across the barrier, only the reagents necessary for nucleic acid amplification can pass through, ensuring that the nucleic acid target remains retained within the small section under normal conditions. Meanwhile, the reagents necessary for nucleic acid amplification (for example, polymerase, buffer components, primers (nucleic acid polymers with a molecular weight below a certain level), and components necessary for the exertion of polymerase enzymatic activity) can be externally added. Therefore, nucleic acids derived from contained cells or cell-like structures can be efficiently amplified. In a preferred embodiment, the gelled droplets, the droplets, or the coated droplets are advantageous structures that are not substantially disrupted by means (agents) or conditions (heating, shearing, and the like) that lyse the cells or cell-like structures. In still another preferred embodiment, the gelled droplets, the droplets, or the coated droplets can suitably used because the gelled droplets, the droplets, or the coated droplets can provide conditions under which the nucleic acid molecule can be homogeneously amplified when the target is a nucleic acid molecule.

In one embodiment, the gelled droplets may be composed of agarose, acrylamide, a photocurable resin, polyethylene glycol, gelatin, sodium alginate, Matrigel, collagen, and hydrogel, and combinations thereof.

In one embodiment, in amplification, amplification is performed while maintaining the gel state within small sections such as gel capsules or gelled droplets. Within this library, nucleic acid molecules in a state suitable for subsequent analysis of nucleic acid sequences can be provided.

When the small section is a gelled droplet, the droplet preferably has a diameter of about 1 to 250 μm. This is because this allows for stable provision as a nucleic acid library. In one embodiment, the diameter when forming the gelled droplets or gel capsules may be about 1 to 250 μm, more preferably about 10 to 200 μm, for example, about 1 μm, about 5 μm, about 10 μm, about 15 μm, about 20 μm, about 25 μm, about 30 μm, about 40 μm, about 50 μm, about 80 μm, about 100 μm, about 150 μm, about 200 μm, or about 250 μm. The diameter of the gel capsule or gelled droplet may be the same as the droplet to be prepared, but the diameter may change upon gelation.

In one embodiment, the large section is a well forming portion of a microplate or a microtube. In the case of the well (forming portion), it can be provided in the form of a multi-well plate including these, and the subsequent processing (for example, preprocessing of sequencing or the like) can be efficiently performed.

In one embodiment, the present disclosure can provide a combination of nucleic acid libraries including a plurality of nucleic acid libraries.

In one preferred embodiment, the present disclosure provides a nucleic acid library having a full-length gene sequence content percentage of 10% or more, 15% or more, 20% or more, 25% or more, 30% or more, 35% or more, 40% or more, 45% or more, or 50% or more. λ nucleic acid library having such a high full-length gene sequence content percentage is not provided in the conventional technology, and the present disclosure can provide a library of very high quality, and can provide data of high quality with higher accuracy and higher coverage in subsequent nucleotide sequence analysis, amino acid sequence analysis, analysis of gene coding sequence, and the like.

In a preferred embodiment, the present disclosure provides a coding region having a length of 1,000 bp or more in a proportion of 3% or more, preferably 5% or more, 10% or more, 15% or more, or 20% or more. A nucleic acid library having such a high percentage of coding regions having a length of 1,000 bp or more is not provided in the conventional technology, and the present disclosure can provide a library of very high quality, and can provide data of high quality with higher accuracy and higher coverage in subsequent nucleotide sequence analysis, amino acid sequence analysis, analysis of gene coding sequence, and the like.

In one aspect, the present disclosure provides a method for preparing a nucleic acid library, the method including: a step of providing two or more cells or cell-like structures; a step of generating a small section containing one or more cells or cell-like structures, from the cells or cell-like structures; a step of lysing the cells or cell-like structures contained in the small section such that the nucleic acids in the cells or cell-like structures are eluted and retained within the small section; a step of bringing the nucleic acids into contact with a nucleic acid amplification reagent to amplify the nucleic acids within the small section and obtain an amplification product, as necessary; and a step of accommodating one or a plurality of the large sections containing the small sections containing the amplification product, in a container for nucleotide sequencing.

In one embodiment, the cells or cell-like structures used are provided in the form of a suspension, but the present disclosure is not limited thereto. While not wishing to be bound by theory, there is an advantage that a suspension facilitates pipetting operations and facilitates suction of a target liquid amount and movement to a microtube or the like, but the present disclosure is not limited thereto.

In one embodiment, preferably, during the amplification, after cells or cell-like structures contained in the small section are lysed, nucleic acids containing the genome in the cells or cell-like structures are eluted into the small section and retained in the small section. While not wishing to be bound by theory, this is because the subsequent nucleic acid amplification can be efficiently performed by being retained in this state.

In one embodiment, it is advantageous that, in the step of obtaining the amplification product, adding a barcode sequence unique to the large section to the nucleic acid is included.

In a further embodiment, in the step of accommodating one or a plurality of the large sections including the small sections containing the amplification product in a container to be subjected to nucleotide sequencing, a plurality of large sections, preferably all of the large sections can be accommodated in such a container, and subsequent nucleotide sequencing can be performed. In this case, by adding a barcode sequence unique to the large section to the nucleic acid, the amplified sequence includes a barcode sequence that enables identification unique to the large section, and then, when the nucleotide sequencing is performed, it is possible to uniquely determine from which large section the sequence is derived. For example, it is also possible to “group” a plurality of large sections, preferably all of the large sections (that is, combining the contents of the large sections into the same container), which allows for more efficient nucleotide sequencing.

In a more preferred embodiment, in the step of accommodating one or a plurality of the large sections including the small section containing the amplification product in a container to be subjected to nucleotide sequencing in the present disclosure, it is advantageous to remove nucleic acids other than nucleic acids amplified to a predetermined length or more. As a result, the contained nucleic acid molecules have substantially a predetermined length or more, and the subsequent analysis of the nucleic acid sequence is efficiently performed. The predetermined length is advantageously, but not limited to, 30 kb or more. Removal of such nucleic acids having a certain length can be achieved, for example, by sorting gel capsules or gelled droplets retaining genomic DNA amplified to a predetermined value or more by a flow cytometer (BD FACSMelody Cell Sorter, BD Biosciences).

In one embodiment, the sample including two or more cells or cell-like structures is a single sample.

In another embodiment, the sample containing two or more cells or cell-like structures may be soil (including seabed soil), seawater, river water, lake water, feces, saliva, skin, sputum, sludge (including activated sludge), industrial wastewater, tissues derived from animals and plants, and surgical irrigation solution.

The number of small sections accommodated in one large section is preferably 2 or more, and more preferably 3 or more, 4 or more, 5 or more, 10 or more, 20 or more, or the like. For example, the number may be 5 to 15, usually 2 to 300, preferably 2 to 50, 5 to 15, 8 to 12, and about 10.

In one embodiment, the number of cells or cell-like structures contained in 1 g of the sample may be 1 million or more, for example, 5 million or more, 10 million or more, 30 million or more, 50 million or more, 100 million or more, 500 million or more, or 1 billion or more, but is not limited thereto. The number of cells may be determined by an image such as a photograph, or may be confirmed with the naked eye through a microscope.

In one embodiment, the cells or cell-like structures may include two or more types, for example, 10,000 types or more, 30,000 types or more, 50,000 types or more, 100,000 types or more, 200,000 types or more, 500,000 types or more, and 1 million types or more. The type of cell can be classified, for example, by interpretation of the identification result by phylogenetic analysis using the 16S rRNA gene.

In one embodiment, one or more or one or more types are 1 to 3 or 1 to 3 types. While not wishing to be bound by theory, having 1 to 3 types is expected to increase the number of genes finally obtained by the same operation, but the present disclosure is not limited thereto.

In another embodiment, one or more or one or more types are 2 to 3 or 2 to 3 types. While not wishing to be bound by theory, having 2 to 3 types is expected to increase the number of genes finally obtained by the same operation, but the present disclosure is not limited thereto.

In the method according to the present disclosure, the step of generating the small sections may be performed by previously containing a component having a gelling ability in the cell or cell-like structure suspension, then flowing the cell or cell-like structure suspension having a gelling ability into a microchannel, and shearing the suspension with off. While not wishing to be bound by theory, this is because in this case, cells or cell-like structures can be efficiently disposed in small sections.

### (Analysis Method)

In one aspect, the present disclosure provides a method for analyzing nucleic acid sequences in cells or cell-like structures, the method including: a step of providing two or more cells or cell-like structures; a step of generating a small section containing one or more cells or cell-like structures, from the cells or cell-like structures; a step of lysing the cells or the cell-like structures contained in the small section such that the nucleic acids in the cells or the cell-like structures are eluted and retained in the small section; a step of obtaining an amplification product by bringing the nucleic acid into contact with the nucleic acid amplification reagent and amplifying the nucleic acid in the small section; a step of accommodating one or a plurality of the large sections including the small sections containing the amplification product, in a container for nucleotide base; a step of determining a nucleotide sequence of the amplification product; a step of analyzing the nucleotide sequence obtained in the above step; a step of analyzing the amino acid sequence encoded by the nucleotide sequence, as necessary; and a step of analyzing a region encoding a gene, as necessary. In a preferred embodiment, the large sections may all be accommodated in the container. This is because all sequencing can be performed at one time.

In the present disclosure, the step of providing two or more cells or cell-like structures may be performed in any manner. The cells may be obtained from a source of cells or may be commercially available.

In the present disclosure, the step of generating a small section containing one or more cells or cell-like structures, from cells or cell-like structures, is realized by separating cells or cell-like structures from a sample containing cells or cell-like structures such as microorganisms such as a bacterium and accommodating the cells or cell-like structures in each small section by a desired number as described As used herein as exemplified in [a] of FIG. 1(C).

In the present disclosure, in the step of lysing the cells or the cell-like structures contained in the small section such that the nucleic acids in the cells or the cell-like structures are eluted and retained in the small section, the cell partition wall of the cells or the cell-like structures may be lysed by immersing a small project such as a droplet encapsulating one cell or cell-like structure in one or more types of lysis reagents. As a result, the cell contents containing genomic DNA can be released to the outside.

In one embodiment, the step of bringing the nucleic acid into contact with the nucleic acid amplification reagent and amplifying the nucleic acid in the small section to obtain an amplification product is achieved by adding the amplification reagent in a state where the nucleic acids are contained in the small section. The amplification reagent contains a primer for DNA to be detected, a label such as a fluorescent substance, an enzyme (heat-resistant DNA polymerase), dNTP, and the like, and contains a reagent used for nucleic acid amplification. The term “fluorescent label” refers to a label that is possessed by an amplified nucleic acid (an amplified product of a target DNA) and emits fluorescence by excitation light, that is, a so-called fluorescent substance. PCR, which is a typical example of the amplification method, is a method for amplifying the target nucleic acid in the sample by repeating a cycle consisting of three steps; (1) DNA denaturation by heat treatment (dissociation from double-stranded DNA to single-stranded DNA), (2) annealing of a primer to a template single-stranded DNA, and (3) elongation of the primer using a DNA polymerase, as one cycle, Annealing and extension may be performed at the same temperature in two steps.

Examples of the fluorescent substance include fluorescent substances (fluorescent dye and quencher) used in a method (probe method) in which a DNA probe in which a fluorescent dye and a quencher are bound to a template DNA in an annealing step, the DNA probe is cleaved in an elongation (reaction) step, and fluorescence from the fluorescent dye suppressed by the quencher is detected. In addition, there is a dye (fluorescent dye) used in a method using a dye that enters between strands of double-stranded DNA and emits fluorescence (intercalator method) or the like. The term “fluorescent label” refers to a label that is possessed by an amplified nucleic acid (an amplified product of a target DNA) and emits fluorescence by excitation light, that is, a so-called fluorescent substance. Examples of the fluorescent substance include fluorescent substances (fluorescent dye and quencher) used in a method (probe method) in which a DNA probe in which a fluorescent dye and a quencher are bound to a template DNA in an annealing step, the DNA probe is cleaved in an elongation (reaction) step, and fluorescence from the fluorescent dye suppressed by the quencher is detected. In addition, there is a dye (fluorescent dye) used in a method using a dye that enters between strands of double-stranded DNA and emits fluorescence (intercalator method) or the like.

In one embodiment, the step of accommodating one or a plurality of the large sections including the small section containing the amplification product in a container to be subjected to nucleotide sequencing, which is performed as necessary, can be realized by a normal method such as sucking the large section containing the small section and arranging the large section in the container.

In another embodiment, the step of determining the nucleotide sequence of the amplification product can be performed using an ordinary known method used in the field, and for example, a next generation sequencer (NGS) can be used. In order to provide a mixture of amplification products derived from a plurality of specimens in a next generation sequencer, a specific artificial nucleic acid sequence including a barcode sequence for identifying a specimen needs to be added to both ends of a nucleic acid fragment for which sequencing is to be performed, and a nucleic acid amplification reaction step therefor is usually performed in addition to the step of amplifying a target nucleic acid sequence.

The step of analyzing the obtained nucleotide sequence can measure various aspects of the nucleotide sequence, and can be performed by analyzing a region encoding a structural gene, analyzing an amino acid sequence encoded by the nucleotide sequence, analyzing a region encoding a gene, and the like.

In one embodiment, a step of grouping a plurality of small sections in a large section into one may be included as necessary. This is because, in this step, a plurality of small sections (for example, droplets) are mixed as one sample and can be subjected to subsequent analysis (for example, sequencing) and the like, and the analysis can be efficiently advanced by subsequent gene sequence acquisition and the like.

In one embodiment, in the step of obtaining the amplification product, adding a barcode sequence unique to the large section to the nucleic acid may be included. Accordingly, using the added barcode sequence as a clue, it is possible to identify which well the amplified sequence is derived from.

In the present disclosure, “grouping” refers to, for example, grouping the sections containing the amplification product (which may be either small sections or large sections) into a smaller number of sections, preferably one section. For example, when grouping small sections, a large section may be used, and when grouping small sections or large sections, combining the sections to another container that is not a large section (for example, a container for nucleotide sequencing) is included. The grouped sections may be the same as or different from the small sections or the large sections.

In one embodiment, the step of obtaining an amplification product or the corresponding amplification reagent accommodation unit includes adding a barcode sequence unique to the large section to the nucleic acid or a reagent for adding the barcode sequence. While not wishing to be bound by theory, this approach allows identification of which large section a sequence is derived from when sequencing is performed simultaneously or at different times after mixing. This enables ultra-parallel information collection.

In a preferred embodiment, the present disclosure includes combining small sections containing an amplification product to a smaller number of sections, and more preferably includes combining small sections containing an amplification product to one section.

In a preferred embodiment, the present disclosure includes accommodating a plurality of large sections, preferably all of the large sections, in one container. More preferably, sorting only nucleic acids amplified to a predetermined length or more for the amplification product is further included. This is because the efficiency of comprehensive sequencing can be increased.

In the preferred embodiment, the step of analyzing the region encoding the genes or the corresponding analysis unit includes calculating a full-length gene sequence percentage. Such a full-length gene sequence percentage has not been taken up as a quality evaluation field of a conventional library, and has been found to be a meaningful evaluation field since the present disclosure has enabled longer and comprehensive sequencing.

In a preferred embodiment, the nucleotide sequencing of the amplification products is performed simultaneously in parallel. In a preferred embodiment, one or a plural number which are the number of targets for simultaneous parallel processing may be 96 or more, 192 or more, 384 or more, 768 or more, 1536 or more, or 3072 or more. In a preferred embodiment, the nucleotide sequencing may be performed using a next generation sequencer.

In one embodiment, the present disclosure provides a method for analyzing a metagenome, the method including: a step of providing two or more cells or cell-like structures that constitute the metagenome; a step of generating a small section containing one or more cells or cell-like structures, from the cells or cell-like structures; a step of lysing the cells or the cell-like structures contained in the small section such that the nucleic acids in the cells or the cell-like structures are eluted and retained in the small section; a step of obtaining an amplification product by bringing the nucleic acid into contact with the nucleic acid amplification reagent and amplifying the nucleic acid in the small section; a step of accommodating one or a plurality of the large sections including the small section containing the amplification product, in a container for nucleotide sequencing, as necessary; a step of determining a nucleotide sequence of the amplification product in the small section; a step of analyzing the nucleotide sequence obtained in the step to obtain nucleic acid sequence information of the metagenome; a step of analyzing the amino acid sequence encoded by the nucleotide sequence, as necessary; and a step of analyzing a region encoding a gene, as necessary. In such a method for analyzing a metagenome, in addition to using a metagenome as a target, a combination of any preferred embodiments described herein can be employed. As a result, metagenomes can be collected more efficiently from various samples.

In another embodiment, in the present disclosure, sorting only nucleic acids amplified to a predetermined length or more for the amplification product is further included. As a representative example of such a technique, there is an example that can be used for analysis of a metagenome.

In another preferred embodiment, the present disclosure provides a method for analyzing a metagenome, the method including: a step of providing two or more cells or cell-like structures that constitute the metagenome; a step of generating a small section containing one or more cells or cell-like structures, from the cells or cell-like structures; a step of lysing the cells or the cell-like structures contained in the small section such that the nucleic acids in the cells or the cell-like structures are eluted and retained in the small section; a step of obtaining an amplification product by bringing the nucleic acid into contact with the nucleic acid amplification reagent and amplifying the nucleic acid in the small section; a step of accommodating one or a plurality of the large sections including the small section containing the amplification product, in a container for nucleotide sequencing, as necessary; a step of determining a nucleotide sequence of the amplification product in the small section; a step of analyzing the nucleotide sequence obtained in the step to obtain nucleic acid sequence information of the metagenome; a step of analyzing the amino acid sequence encoded by the nucleotide sequence, as necessary; and a step of analyzing a region encoding a gene, as necessary. In this analysis method, any features described in (Nucleic Acid Library and Method for Preparing the Same) can be appropriately combined and used.

In one embodiment, the analysis method of the present disclosure includes a step of simultaneously and in parallel determining nucleotide sequences of amplification products in small sections grouped into one in a large section, and a step of analyzing the nucleotide sequence obtained in the above step using a computer and estimating a region coding for a protein to obtain a gene.

In certain embodiments, the plurality may be 96 or more, 384 or more, 768 or more, 1,152 or more, 1,536 or more, 1,920 or more, 2,304 or more, 2,688 or more, 3,072 or more, and the like.

In another aspect, the present disclosure provides an analysis system for a nucleic acid sequence in cells or cell-like structures, the method including: a small section generation unit that generates a small section containing one or more cells or cell-like structures, from the cells or cell-like structures; a nucleic acid lysis unit that lyses the cells or the cell-like structures contained in the small section such that the nucleic acids in the cells or the cell-like structures are eluted and retained in the small section; an amplification reagent accommodation unit that accommodates a nucleic acid amplification reagent used for obtaining an amplification product by coming into contact with the nucleic acid and amplifying the nucleic acid in the small section; an accommodation unit that accommodates one or a plurality of the large sections including the small sections containing the amplification product, as necessary; a nucleotide sequencing unit that determines a nucleotide sequence of an amplification product in the small section; a nucleotide sequence analysis unit that analyzes the nucleotide sequence; an amino acid sequence analysis unit that analyzes an amino acid sequence encoded by the nucleotide sequence, as necessary; and a coding region analysis unit that analyzes a region encoding a gene, as necessary. This analysis system can appropriately combine and use any features described in (Nucleic Acid Library and Method for Preparing the Same).

In one embodiment, the small section generation unit that generates a small section containing one or more cells or cell-like structures, from cells or cell-like structures, included in the system of the present disclosure may be any unit as long as the system for manufacturing a small section is included. For example, it may be a device or means for manufacturing gelled droplets, droplets, or coated droplets. This can be performed by encapsulating microbial cells in droplets. For example, in the case of a microdroplet, a microchannel is exemplified, and a microdroplet accommodating a desired number of cells can be prepared by flowing a suspension of microbial cells into the microchannel and shearing the suspension. The shearing may be performed at regular intervals. Shearing of the suspension can be performed using oil. As the oil, for example, a mineral oil (for example, light mineral oil), a vegetable oil, a silicone oil, or a fluorinated oil can be used, and thus means for performing such shearing may be provided.

The nucleic acid lysis unit that dissolves the cells or the cell-like structures contained in the small sections such that the nucleic acids in the cells or the cell-like structures are eluted and retained in the small sections may be any means as long as the nucleic acid lysis unit can provide conditions that the nucleic acid is eluted and retained in the small sections, such as heating the small sections.

The amplification reagent accommodation unit that accommodates a nucleic acid amplification reagent used for obtaining an amplification product by coming into contact with the nucleic acid and amplifying the nucleic acid in the small section may be any unit as long as the amplification reagent can be accommodated.

In one embodiment, the accommodation unit that accommodates one or a plurality of the large sections including the small section containing the amplification product may be, for example, a pipette as long as the small sections can be collected by suction or the like and moved to the large sections.

In one embodiment, the nucleotide sequencing unit that determines the nucleotide sequence of the amplification product in the small section may be any unit that can be sequenced, and may be a next generation sequencer (NGS) or the like.

The nucleotide sequence analysis unit that analyzes the nucleotide sequence may be any unit as long as the read nucleotide sequence can be analyzed, but may be any unit as long as the nucleotide sequence can be input and analyzed by a computer or the like.

The amino acid sequence analysis unit that analyzes the amino acid sequence encoded by the nucleotide sequence may be any unit as long as the amino acid sequence can be analyzed by a computer or the like.

The coding region analysis unit that analyzes a region encoding a gene may be any unit as long as a nucleic acid sequence can be analyzed and a component corresponding to a structural gene can be analyzed as long as the analysis can be performed by a computer or the like.

Analysis processing of a nucleotide sequence, an amino acid sequence, and a region encoding a gene can be performed by using SPAdes, which is a tool used for processing of connecting read nucleotide sequence fragments, or Prokka, which is a tool for estimating a region encoding a gene, and referring to a nucleotide sequence, an amino acid sequence database, or the like as necessary, but the analysis processing is not limited thereto, and any application other than these may be used. The analysis unit that analyzes each of the nucleotide sequence, the amino acid sequence, and the gene may be the same analysis unit, and for example, may be capable of analyzing two or three or more functions thereof.

The method and system of the present disclosure may be provided with a computer program for realizing the same, and the present disclosure also provides such a program.

In one embodiment, the nucleotide sequencing of the amplification products is performed simultaneously in parallel. Such a method can be realized by performing the nucleotide sequence of the amplification product in the small section grouped into one in the large section. In this case, since the barcode sequence is included, sequencing can be performed simultaneously in parallel, and ultra-parallel processing is achieved.

In another aspect, the present disclosure provides a method for preserving nucleic acid molecules, the method including a step of further preserving a large section accommodating small sections as it is or after adding a substance inhibiting DNA degradation at room temperature or less, after the final step of the method for preparing a nucleic acid library according to the present specification.

The nucleic acid molecules in the present disclosure are from microorganisms. In one embodiment, in the preserving method of the present disclosure, the temperature which is equal to or lower than room temperature is 4 °C or lower, preferably −20 °C or lower, and more preferably −80 °C. or lower.

In one embodiment, in the present disclosure, examples of a method for separating cells such as bacteria from a sample such as a microbiota obtained in the above step and accommodating a desired number of cells in each section include, but not limited to, (1) a method using a microchannel, (2) a method for fluorescently labeling cells and subsequently accommodating the cells using a flow cytometer (fluorescence-activated cell sorter (FACS), (3) a method using a micromanipulator capable of micromanipulating microbial cells under microscopic observation, and (4) a limiting dilution method of stochastically obtaining a single cell.

In addition, when a desired number of cells from a sample such as a microbiota are accommodated in each small section, a buffer solution containing a salt, a nutrient, other components, or the like can be used as a medium for suspending the cells, in addition to physiological saline. For example, when droplets are employed as small sections, any component may be used as long as the component is suitable for droplet generation. Examples of such a buffer solution include PBS, Tris-HCl, TE, HEPES, and the like, and besides, sterilized water, sea water, artificial sea water, various liquid media, and the like can be mentioned, but the present disclosure is not limited thereto. Media such as water or buffer without surfactant may be preferred to generate droplets.

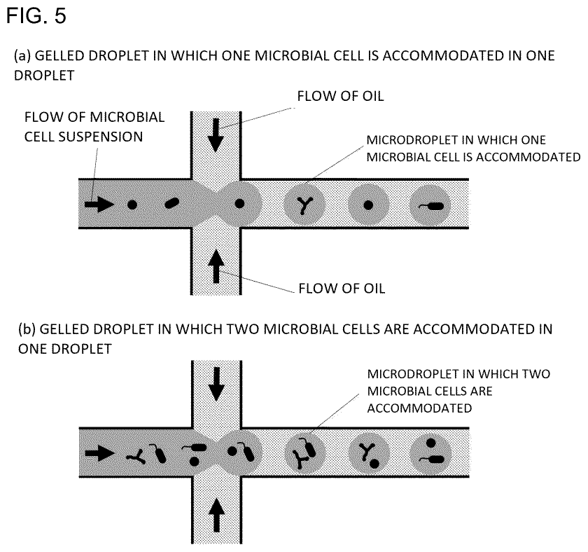

The following describes a method for preparing microdroplets encapsulating single cells using a microchannel with reference to FIG. 5(a) and a method for preparing microdroplets encapsulating double cells with reference to FIG. 5(b). Here, the term “microdroplet” refers to a very small droplet having a volume of nanoliters (10⁻⁹ L) to picoliters (10⁻¹² L) or less, which are prepared using microfluidics technology. This technology involves the mixing, separation, and detection of liquid reagents on a miniature device that forms micrometer-sized microchannels or reaction chambers on a silicon or glass substrate.

The step of accommodating one or two cells per small section in the small section can be performed by encapsulating one or two cells in a droplet. For example, when microdroplets are prepared using a microchannel, a suspension of cells is caused to flow into the microchannel, and the suspension is sheared, whereby microdroplets accommodating one or two cells can be prepared. The shearing may be performed at regular intervals. Shearing of the suspension can be performed using oil. As the oil, for example, a mineral oil (for example, light mineral oil), a vegetable oil, a silicone oil, or a fluorinated oil can be used.

Here, by adjusting the cell concentration of the suspension, the number of microbial cells encapsulated per droplet can be adjusted. In the case of the condition for generating 70,000 droplets per microliter, setting the cell concentration of the suspension to 7,000 to 14,000 cells/μL results in a theoretical value of 0.1 to 0.2 cells/droplet. This allows 90% or more of the droplets that actually encapsulate cells to contain a single cell. Similarly, by adjusting the cell concentration of the suspension to a theoretical value of 2 cells/droplet, 31% or more of the droplets that encapsulate cells can contain two cells. In addition, by adjusting the cell concentration of the suspension to a theoretical value of 3 cells/droplet, 23% or more of the droplets that encapsulate cells can contain three cells.

The diameter of the droplets may be about 1 to 250 μm, more preferably about 10 to 200 μm, and most preferably about 20 to 60 μm. For example, the diameter of the droplets may be about 1 μm, about 5 μm, about 10 μm, about 15 μm, about 20 μm, about 30 μm, about 40 μm, about 50 μm, about 60 μm, about 70 μm, about 80 μm, about 90 μm, about 100 μm, about 150 μm, about 200 μm, or about 250 μm.

The method for preparing a metagenomic library of the present disclosure can include, in a case where gelled droplets are employed as small sections, a step of gelling the above-generated droplets to generate gelled droplets. The gelling of the droplets can be performed, for example, by incorporating a gelling material that gels upon cooling to a predetermined temperature and configuring the droplets, then cooling the prepared droplets to the predetermined temperature. Alternatively, the gelling can also be performed by incorporating a gelling material that gels upon light irradiation and configuring the droplets, then applying light stimulation to the prepared droplets. The droplets can contain a material for gelled droplets, for example, by including a material for gelling (also referred to as a gelling material) in the suspension of microbial cells. Examples of the gelling material include agarose (including low-melting-point agarose), acrylamide, a photocurable resin (for example, PEG-DA), PEG, gelatin, sodium alginate, Matrigel, and collagen. The gelled droplets may be hydrogelled droplets. In the present disclosure, the term “hydrogel” refers to a substance in which a solvent or a dispersion medium retained by a network structure of polymer substances or colloidal particles is water.

In this technical field, when extracting DNA from a large amount of cells at once, a method for preparing DNA using phenol-chloroform extraction and ethanol precipitation is common. However, when the acquisition and analysis of genetic material from one to several cells is contemplated, the amount of genetic material obtained from the cells is very small and needs to be converted into a nucleic acid-only state without loss. For one to several cells, attempting nucleic acid purification using a general bulk scale procedure may result in either no nucleic acid being extracted at all or the loss of most of the nucleic acid during the step of separation and purification from contaminants. However, by using gelled droplets encapsulating one or several cells or cell-like structures, the purified genetic material (for example, DNA) can be retained within the gelled droplets, while also eliminating the possibility of contamination from external molecules. In addition, in terms of operation, a large number of one or several cells can be processed in parallel as a whole with a very simple operation. Specifically, contaminants can be removed by performing a step of centrifuging a solution containing gelled droplets that encapsulate one to several cells, removing the supernatant, and replacing with a washing solution. Alternatively, a step of filtering the gelled droplets with a filter, removing the supernatant, allowing a washing solution to pass through, and finally recovering the gelled droplets can also be performed. By using the gelled droplets, residual reagents and the like can be diluted while retaining genetic material. This step can also be repeated. By adding this step, the subsequent operation, for example, the amplification reaction can be smoothly performed.

### [1]-1 Generation of Small Section

As used herein, the step of accommodating a desired number of cells or cell-like structures such as microorganisms in each small section is realized by separating cells or cell-like structures from a sample containing cells or cell-like structures such as microorganisms such as a bacterium and accommodating the cells or cell-like structures in each small section by a desired number as exemplified in [a] of FIG. 1(C). The desired number is 1 to 3 or 2 to 3, more preferably 1 to 3, and most preferably 1. Hereinafter, a specific example of a step of accommodating a desired number of microorganisms in each small section will be described, but the method is not limited thereto. First, microbiota is separated from a sample containing microorganisms such as bacteria. Here, the term “microbiota” means a collection of microorganisms contained in a sample containing microorganisms such as bacteria. For example, 5 g of a sample is suspended in an aqueous solution such as 6 ml of physiological saline, a buffer solution, or sterilized water, and allowed to stand to remove contaminants by precipitation, and then microorganisms such as bacteria present in the supernatant are centrifuged at 8,000 to 10,000× g, and the precipitate is recovered, whereby a microbiota can be obtained. The precipitate containing the microbiota obtained here is further suspended in an aqueous solution, and then centrifugation and precipitate recovery are repeated several times, whereby a microbiota with less contaminants can be obtained. The number of microbial cells contained in the microbiota is any number of 2 or more, and may be, for example, 10 or more, 50 or more; 100 or more, 500 or more, 1,000 or more, 5,000 or more, 10,000 or more, 50,000 or more, 100,000 or more, 500,000 or more, 1 million or more, 5 million or more, or 10 million or more, but is not limited thereto. Examples of the sample containing microorganisms such as bacteria include soil (including seabed soil), sea water, river water, lake water, feces, saliva, skin, sputum, sludge (including activated sludge), industrial wastewater, tissues derived from animals and plants, and surgical irrigation solutions, but are not limited thereto.

### [1]-2 Lysis

In the present disclosure, the lytic step performed as necessary can be performed, for example, as shown in [b] of FIG. 1(C), by immersing a gelled droplet encapsulating one cell or cell-like structure in one or more types of lysis reagents to lyse the cell partition wall of the cell or cell-like structure, and releasing the cell content containing genomic DNA to the outside. In the present disclosure, the term “cell partition wall” broadly includes a membrane and/or a wall constituting a barrier separating the inside and the outside of cells or cell-like structures, in the cell or the cell-like structure. The structure of the cell partition wall greatly differs depending on whether the cell or the cell-like structure corresponds to a bacterium, an archaea, a fungus, an animal cell, a plant cell, a virus, or the like. For example, in the case of bacteria, the cell partition wall is composed of a cell membrane and a cell wall, the cell membrane is composed of a phospholipid bilayer, and the cell wall present outside the cell membrane is mainly composed of peptidoglycan. Also in the case of fungi, the cell partition wall is composed of a cell membrane and a cell wall, but the cell membrane contains ergosterol in addition to a phospholipid bilayer, and the cell wall is mainly composed of glucan, chitin, mannan, and the like, unlike bacteria. In the case of animal cells, the cell partition wall is composed only of a cell membrane, and the cell membrane is mainly composed of phospholipids and cholesterol. As the lysis reagent for lysing the cell partition walls of various types of cells or cell-like structures, enzymes, surfactants, other denaturants, reducing agents, and pH adjusting agents can be used in combination.

More specifically, the lysis reagent may contain at least one selected from the group consisting of lysozyme, labiase, yatalase, achromopeptidase, protease, nuclease, zymolyase, chitinase, lysostaphin, mutanolysin, sodium dodecyl sulfate, sodium lauryl sulfate, potassium hydroxide, sodium hydroxide, phenol, chloroform, guanidine hydrochloride, urea, 2-mercaptoethanol, dithiothreitol, TCEP-HCL, sodium cholate, sodium deoxycholate, Triton X-100, Triton X-114, NP-40, Brij-35, Brij-58, Tween 20, Tween 80, octyl glucoside, octyl thioglucoside, CHAPS, CHAPSO, dodecyl-β-D-maltoside, Nonidet P-40, and Zwittergent 3-12. More preferably, at least one selected from the group consisting of lysozyme, achromopeptidase, protease, sodium dodecyl sulfate, and potassium hydroxide may be used as the lysis reagent.

The immersion time of the gelled droplet varies depending on the type and concentration of the lysis reagent to be used, but for example, when microbial cells in the gelled droplet are lysed by sequentially adding 50 U/μL lysozyme, 1 mg/mL protease K, and 0.5% sodium dodecyl sulfate (SDS) 0.5%, lysis can be reliably performed by allowing all the lysis reagents to stand at 37 °C for 1 hour, preferably 2 hours after being added.

When nucleic acid is amplified or analyzed for each cell for various microbial cell groups, for example, gram-positive bacteria have a cell wall including a thick peptidoglycan layer, and thus there is a possibility that cells cannot be sufficiently lysed only with a mild one, and it is desirable to use a somewhat strong one as a lysis reagent or a combination of lysis reagents.

### [1]-3 Nucleic Acid Extraction

In the present disclosure, as shown in [c] of FIG. 1(C), the extraction of the nucleic acid (for example, DNA) means a step of removing substances other than polynucleotides constituting the nucleic acid such as genomic DNA, such as cell partition wall remnants and contaminants such as proteins binding to the nucleic acid such as genomic DNA, from the cell lysate obtained by the previous step as necessary, and creating a state where the nucleic acid such as genomic DNA derived from cells is present in the section in a state of high purification degree. By this step, the subsequent operation, for example, the amplification reaction can be smoothly performed.

The lysis reagent used in the above-mentioned lysis step, there is a possibility that the remnants of cell partition walls, contaminants such as proteins that bind to nucleic acids such as genomic DNA, and the like may inhibit a reaction such as nucleic acid amplification of DNA and the like, and it is preferable that they are sufficiently removed from the inside of gelled droplets before the subsequent steps. The nucleic acid such as genomic DNA eluted in the gelled droplets in the lysing step is retained in the gelled droplets and does not leak out of the gelled droplets even when the gelled droplets are immersed in each solution. Meanwhile, contaminants such as a lysis reagent remaining in the gelled droplets, a remnant of a cell partition wall, and a protein bound to the nucleic acid such as genomic DNA can leak out of the gelled droplets. Therefore, by repeating the step of immersing the gelled droplets after the lysing step in a washing solution, centrifuging the gelled droplets, removing the supernatant, and replacing the gelled droplets with a new washing solution, contaminants such as a lysis reagent can be removed. Alternatively, in the step of filtering the gelled droplets with a filter, removing the supernatant, allowing a washing solution to pass through, and finally recovering the gelled droplets, the contaminants such as lysis reagents can also be removed.

The use of a strong lysis reagent or a combination of lysis reagents can enable comprehensive amplification of nucleic acids or analysis of nucleic acids such as genomes, regardless of the type of various cells (including those having a cell wall and other types of microorganisms). Since only one molecule of genomic DNA is usually present in a cell, it is important that the cell is completely lysed and the proteins bound to the genomic DNA are sufficiently removed in the subsequent steps when the entire region of the genomic DNA is intended to be amplified without leakage. As a result, even when a specimen composed of several hundred or more types of microorganisms such as intestinal microorganisms is targeted, it is possible to completely lyse all of them homogeneously and amplify the entire genome region from all of them without omission. In addition, this makes it possible to prepare a high-quality genomic library, and finally obtain high-quality and highly efficient gene sequence information.

### [1]-4 Nucleic Acid Amplification

In the present disclosure, nucleic acid amplification means a step of amplifying a nucleic acid (for example, whole genomic DNA) using a target nucleic acid molecule such as genomic DNA extracted in the previous step as a template, as shown in [d] of FIG. 1(C). This step is useful when the amount of nucleic acid (for example, DNA) in the large section is extremely small, and can be omitted when the amount is sufficient for the amount of nucleic acid (DNA or the like) in the large section. This step is a step which is beneficial for obtaining a sufficient amount of single amplified genome (SAG) from an extremely small amount of genomic DNA in the femtogram (10-15) range per cell, enabling sequence analysis by a next generation sequencer. The step of amplifying the nucleic acid includes whole genome amplification (WGA).

Examples of the WGA include a multiple displacement amplification (MDA) method, a multiple annealing and looping baced amplification cycles (MALBAC) method, and a degenerate oligonucleotide-primed PCR. (DOP-PCR) method. Among these methods, the MDA method is a method in which a DNA replication reaction (also referred to as a thermostatic strand displacement amplification reaction) is performed under a thermostatic reaction condition at 30 °C using phi29 DNA polymerase and a random primer, it is possible to replicate long-chain DNA of kilobase or more by highly accurate DNA replication and strand displacement activity by phi29 DNA polymerase, and finally, it is possible to obtain a genome amplification product in a microgram amount from a nanogram from femtogram level genomic DNA. The MDA method can be suitably used in the step of amplifying genomic DNA in the present disclosure.

Note that, for example, when a gelled droplet is employed as a small section, there is a possibility that a reaction involving a heat treatment may cause re-lysing of the gel (for example, agarose gel), and the shape of the small-sectioned gelled droplet may be disintegrated to disable the isolation of the contents of the gelled droplet. Therefore, when the enzymatic reaction is allowed to proceed in the gelled droplet, it is desirable to use an enzyme or a reaction system in which the enzymatic reaction can proceed at a temperature equal to or lower than the melting temperature in accordance with the melting temperature of the gelling material. For example, the MDA method can be suitably employed, and examples of the enzyme that can be used include, but are not limited to, the phi29 polymerase (optimum temperature: 30 °C), the recombinase polymerase (optimum temperature: 37 to 42 °C), and the Bst polymerase (optimum temperature: 60 °C to 65 °C).

Furthermore, in the present disclosure, sequencing can be performed after the large sections are grouped into one container in a stage where the large sections accommodating one or a plurality of small sections containing amplification products are subjected to sequencing. In that case, in order to determine from which large section the obtained nucleotide sequence is derived, a unique barcode sequence (also referred to as a nucleic acid barcode) can be added for each large section after this amplification step. The length of the barcode sequence may be 4 to 12 bases, preferably 6 to 10 bases, and most preferably 8 to 10 bases. Two or more types of nucleic acid barcodes may be used in combination, and according to the combination, for example, even when 8 microplates having 384 wells are simultaneously used in parallel (3,072 wells in total), it is possible to distinguish each well by using 3,072 types of nucleic acid barcodes having different sequences for each well. When the number of types or combinations of DNA barcodes to be prepared is further increased, ultra-parallel processing using still more wells can be supported. Note that, when adding the nucleic acid barcode molecules for identifying the position of the large section, it is important to know which sequence of the nucleic acid barcode molecule was added to which position of the well.

[1]-5 Accommodation of Small Section into Large Section

In the present disclosure, the accommodation of the small sections into the large sections is realized by accommodating one or a plurality of small sections containing the genomic DNA amplification fragments obtained in the previous step in the large sections as in [e] of FIG. 1(C). As the large section, as described above, a well of a microplate or a microtube can be used. The step can be performed, for example, in a case where the small sections are gelled droplets, by centrifugally washing the gelled droplets after nucleic acid amplification with PBS as a washing solution, then staining with a DNA intercalator with a fluorescent dye (for example, SYBR green (5760A, Takara Bio Inc.), EvaGreen (31000, Cosmo Bio Co., Ltd.)), sorting the gelled droplets containing genomic DNA amplified to a predetermined amount or more using a flow cytometer, and accommodating a predetermined number of the gelled droplets (small sections) in each well (large sections) of the microplate (for example, HSP3801, Bio-Rad Laboratories, Inc.). In addition, in this step, while counting with naked eyes using a micropipette, a predetermined number of gelled droplets can be manually accommodated in the microtubes as large sections. The step of accommodating a predetermined number of gelled droplets in each of the large sections (for example, a well) can also be performed by dispensing equal volumes based on a previously determined droplet concentration. For example, by preparing a gelled droplet suspension to a droplet concentration of 20 droplets/100 μL and dispensing the gelled droplet suspension into 50 μL of each large section (for example, a well), a nucleic acid library such as a metagenome, in which 10 gelled droplets per large section (for example, a well) are accommodated, can be prepared. Here, the number of small sections to be accommodated in the large section is 2 to 300, preferably 2 to 50, more preferably 5 to 15, still more preferably 8 to 12, and most preferably 10.

In one embodiment, the steps from the introduction of cells or cell-like structures into the small sections to the amplification and determination of the nucleic acid sequence can be carried out, for example, by the following technique. For example, there is provided a method for amplifying polynucleotides in cells or cell-like structures, including: a step of encapsulating the cells or cell-like structures in one or a plurality of cells or structure units or droplets, using a sample containing the cells or cell-like structures; a step of gelling the droplets to generate gel capsules or gelled droplets; a step of immersing the gel capsules or gelled droplets in one or more types of lysis reagents to lyse the cells or cell-like structures, that is, a step in which the polynucleotides in the cells are eluted into the gel capsules or gelled droplets and are retained in the gel capsules or gelled droplets in a state where substances binding to the polynucleotides have been removed; and a step of bringing the polynucleotides into contact with amplification reagents to amplify the polynucleotides within the gel capsules or gelled droplets.

In one embodiment, the method includes a step of sorting gel capsules or gelled droplets in which the polynucleotides are amplified one by one and separately collecting the gel capsules or gelled droplets.

In one embodiment, the cells may include microbial cells.

In one embodiment, after the gel capsules or the gelled droplets are immersed in the lysis reagent, the lysis reagent and contaminants are removed from the gel capsules or the gelled droplets.

In another embodiment, a suspension of cells or cell-like structures is flowed into a microchannel, and the suspension is sheared with oil to prepare the droplets encapsulating the cells or cell-like structures.

In one embodiment, the gel capsules or gelled droplets are hydrogel capsules or gelled droplets.

In one embodiment, the amplifying step is performed by a thermostatic strand displacement amplification reaction.

In one embodiment, the system of the present disclosure may include: a droplet preparation unit that encapsulates cells or cell-like structures in one cell or structure unit or droplet; and a gel capsule or gelled droplet generation unit that gels droplets to generate gel capsules or gelled droplets.

A lysis reagent immersion unit that immerses the gel capsules or the gelled droplets in a lysis reagent; a removing unit that removes contaminants from the gel capsules or the gelled droplets; and an amplification reagent immersion unit that immerses the gel capsules or the gelled droplets in an amplification reagent may be provided.

In one embodiment, a sequencing unit that performs sequencing of a nucleic acid sequence in the polynucleotide amplified in the amplification reagent immersion unit is further provided, and the genome sequence of the cell is determined at a single cell level. In one embodiment, a sorting unit that sorts gel capsules or gelled droplets and accommodates the gel capsules or gelled droplets in a storage container is further provided, and is for preparing a metagenomic library. In one embodiment, the droplet preparation unit includes a microchannel.

In one embodiment, a method for amplifying polynucleotides in cells or cell-like structures of the present disclosure, may include: a step of encapsulating the cells or cell-like structures in one cell or structure unit or droplets, using a sample containing two or more cells or cell-like structures (for example, viruses, organelles such as Mt or Nuc); a step of gelling the droplets to generate gel capsules or gelled droplets; a step of immersing the gel capsules or gelled droplets in one or more types of lysis reagents to lyse the cells or cell-like structures, that is, a step in which the polynucleotides in the cells are eluted into the gel capsules or gelled droplets and are retained in the gel capsules or gelled droplets in a state where substances binding to the polynucleotides have been removed; and a step of bringing the polynucleotides into contact with amplification reagents to amplify the polynucleotides within the gel capsules or gelled droplets.

In one embodiment, the amplification methods used in the present disclosure are capable of individually amplifying a genome or similar gene assembly at the so-called single cell level. The amplification method of the present invention realizes individual genome amplification by a very simple method. Therefore, nucleic acid information can be obtained at one time for cells of 100 units, 1,000 units, 10,000 units, 100,000 units, or more, and thus a library can also be obtained.

In one embodiment, the number of cells or cell-like structures that can be targeted in the amplification method of the present invention is any number of 2 or more, and may be, for example, 10 or more, 50 or more, 100 or more, 500 or more, 1,000 or more, 5,000 or more, 10,000 or more, 50,000 or more, 100,000 or more, 500,000 or more, 1 million or more, 5 million or more, or 10 million or more. The amplification methods used in the present disclosure can target a larger number of cells compared to a conventional single cell reaction system, for example, a 0.2 mL or 1.5 mL microtube reaction system.

The cells or the cell-like structures targeted by the amplification method used in the present disclosure can include any one of those described in the section (Cells and Cell-like Structures). In one preferred embodiment, cells may be targeted. In another embodiment, a cell-like structure is targeted, and among them, a virus, a cell organ such as mitochondria or nuclei, or the like can be targeted.

In the amplification method used in the present disclosure, a sample containing provided cells or cell-like structures may be provided in any form. As the medium contained in the sample, an appropriate medium (containing buffers, salts, nutrients, other components, and the like) can be selected for any cell or cell-like structure selected from the section (Cells and Cell-like Structures). As such a component, any component may be used as long as the component is suitable for droplet generation. It is also preferable that the component is an appropriate component at the time of gelling. Examples of such components include, but are not limited to, sterilized water, sea water, artificial sea water, various liquid media, and the like, in addition to the buffer solution such as PBS, Tris-HCl, TE, HEPES, and the like. Media such as water or buffer without surfactant may be preferred to generate droplets.

As used herein, the encapsulation of cells or cell-like structures into a droplet of one cell or structure unit can employ any embodiment described in the section (Droplet Preparation). Typically, by using a microchannel, a suspension of cells or cell-like structures is caused to flow into the microchannel, and the suspension is sheared, a droplet encapsulating one cell or cell-like structure can be prepared. In addition to the description herein, those skilled in the art can appropriately prepare components and parameters and carry out the preparation with reference to representative examples exemplified in Examples.

In one embodiment, in the amplification method used in the present disclosure, the step of gelling the droplets to generate gel capsules or gelled droplets is gelled or the like, and the gelling can be performed by cooling the prepared droplets configured such that the droplets or droplet materials (for example, a sample containing cells or cell-like structures) contain materials for forming gel capsules or gelled droplets, or the gelling can be performed by applying a stimulus such as light.

Here, in the step of lysing the cells or the cell-like structures, it may be advantageous that the polynucleotides from the cells are processed to be eluted into the gel capsules or gelled droplets and retained within the gel capsules or gelled droplets, in a state where substances bound to the polynucleotides have been removed. As described above, in order to maintain the state where the substance bound to the polynucleotides is removed, it is necessary to reliably destruct the cell wall/cell membrane structure of the cells or the cell-like structures and denature the protein contained in the cell and the substance bound to the polynucleotide by adding various types of lysing agents stepwise or simultaneously. Lysis is achieved by adding reagents stepwise from destruction of the outer cell layer. Furthermore, since the lysates remaining in the gel capsules or the gelled droplets after the lysing operation and the lysis reagents inhibit polynucleotide amplification in the subsequent stage, it may be desirable to cause the lysates and the lysis reagents to pass through the gel capsule or the gelled droplet using an appropriate washing solution, and release the inhibitor to the outside of the gel capsules or the gelled droplets. In order to complete these operations in gel capsules or gelled droplets, it may be desirable to adopt a hydrogel structure that achieves the penetration and release of various chemical solutions and cell lysates while retaining polynucleotides in the gel capsules or gelled droplets. By using the gel capsules, residual reagents can be diluted while retaining genetic material. This step can also be repeated. By diluting the reagent to a level at which inhibition does not occur, a downstream operation, for example, an amplification reaction can be smoothly performed.

### (Gelling)

The present disclosure may include gelled droplets to generate gel capsules. In addition, in the present invention, the device may include a gel capsule generation unit for generating gel capsules by gelled droplets. Gelling of the droplets can be performed by cooling the prepared droplets configured to contain the material of the gel capsule in the droplets. Alternatively, gelling can be performed by giving a stimulus such as light to the droplets. The material of the gel capsules can be included in the droplets, for example, by including the material of the gel capsules in a suspension of cells or cell-like structures. In one embodiment, the gel capsules may be hydrogel capsules.

In one embodiment, when extracting DNA from this large amount of cells at once, it is possible to purify DNA using phenol-chloroform extraction and ethanol precipitation. However, when the acquisition and analysis of genetic material from a single cell is contemplated, the amount of genetic material per cell is very small and needs to be individually converted into a nucleic acid-only state without loss. For a single cell, attempting nucleic acid purification using a general bulk scale procedure may result in either no nucleic acid being extracted at all or extraction of only nucleic acid derived from contaminants. Contamination and loss of target genetic material are major problems in single-cell experiments. However, by using gel capsules encapsulating a single cell or cell-like structure, the purified genetic material (for example, DNA) can be retained within the gel capsule, while also eliminating the possibility of contamination from external molecules. In addition, in terms of operation, a large amount of single cells can be processed in parallel with a very simple operation. A step of centrifuging the test tube containing the gelled droplets, removing the supernatant, and replacing with a washing solution can be performed. Alternatively, a step of filtering the gelled droplets with a filter, removing the supernatant, allowing a washing solution to pass through, and finally recovering the gel capsule can also be performed. By using the gel capsules, residual reagents can be diluted while retaining genetic material. This step can also be repeated. By diluting the reagent to a level at which inhibition does not occur, a downstream operation, for example, an amplification reaction can be smoothly performed.

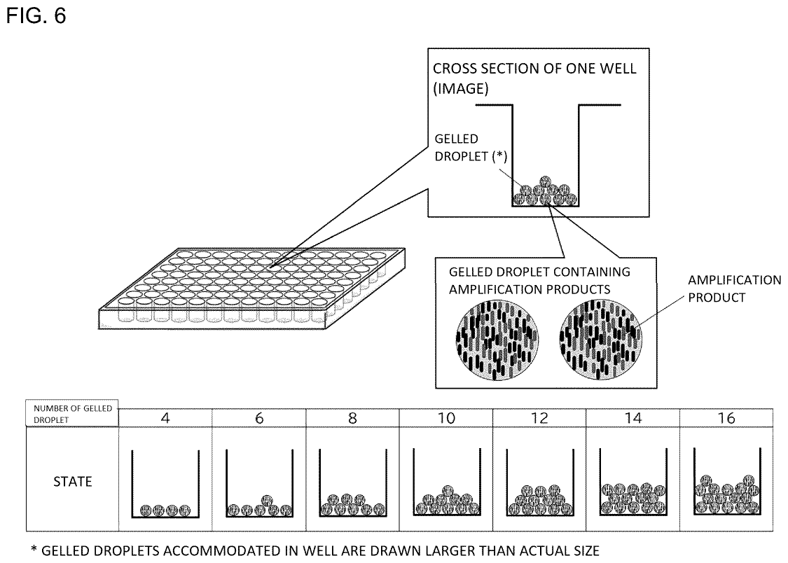

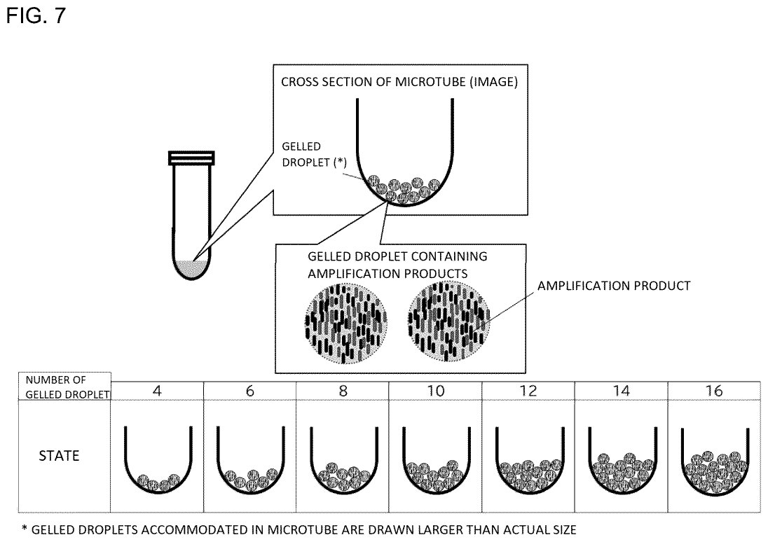

In this manner, the metagenomic library of the present disclosure in which a predetermined number of small sections containing the amplified genomic DNA are accommodated in the large sections is completed. FIG. 6 shows an image of a metagenomic library prepared using a microplate, and FIG. 7 shows an image of a metagenomic library prepared using a microtube. In the lower part of FIGS. 6 and 7, a side view of a well (large section) accommodating 2, 4, 6, 8, 10, 12, 14, and 16 gelled droplets (small sections) and a side view of a tube are shown.

In addition, in the present disclosure, in one aspect, one large section accommodating a predetermined number of small sections as described above may be referred to as a metagenomic library of the present disclosure, and in another embodiment, a plurality of large sections, which are grouped and accommodates a predetermined number of small sections, may be referred to as a metagenomic library of the present disclosure. As the metagenomic library in which a plurality of large sections are grouped, there may be a metagenomic library consisting of 384 large sections, a metagenomic library consisting of 768 large sections, a metagenomic library consisting of 1,152 large sections, a metagenomic library consisting of 1,536 large sections, a metagenomic library consisting of 1,920 large sections, a metagenomic library consisting of 2,304 large sections, a metagenomic library consisting of 2,688 large sections, a metagenomic library consisting of 3,072 large sections, and the like, but the metagenomic library is not limited thereto, and it is possible to increase or decrease the number of large sections without limitation depending on the properties of the sample (diversity or the number of types of cells or cell-like structures included in the sample) and the like.

### [1]-6 “Grouping” a Plurality of Large Sections

In one embodiment, the present disclosure may include a step of accommodating one or a plurality of the large sections including the small section containing the amplification product in a container to be subjected to nucleotide sequencing as necessary. The number of large sections to be accommodated may be one or two or more, and all prepared large sections (denoted here as n, where n is an integer of 2 or greater) may also be accommodated in one container (referred to herein as “grouping”). The grouping number may be any integer of 2, 3 . . . n−1, and n.

Even when a plurality of large sections are grouped, it is advantageous to provide means for determining which large section was used when the subsequent nucleotide sequencing is performed. Such examples may include, but are not limited to, adding barcode sequences unique to large sections to the nucleic acids. Accordingly, using the added barcode sequence as a clue, it is possible to identify which well the amplified sequence is derived from. Furthermore, in the present disclosure, sequencing can be performed after the large sections are grouped into one container in a stage where the large sections accommodating one or a plurality of small sections containing amplification products are subjected to sequencing. In that case, in order to determine from which large section the obtained nucleotide sequence is derived, a unique barcode sequence (also referred to as a nucleic acid barcode) can be added for each large section after this amplification step. The length of the barcode sequence may be 4 to 12 bases, preferably 6 to 10 bases, and most preferably 8 to 10 bases. Two or more types of nucleic acid barcodes may be used in combination, and according to the combination, for example, even when 8 microplates having 384 wells are simultaneously used in parallel (3,072 wells in total), it is possible to distinguish each well by using 3,072 types of nucleic acid barcodes having different sequences for each well. When the number of types or combinations of DNA barcodes to be prepared is further increased, ultra-parallel processing using still more wells can be supported. Note that, when adding the nucleic acid barcode molecules for identifying the position of the large section, it is important to know which sequence of the nucleic acid barcode molecule was added to which position of the well. The term “ultra-parallel”, that is, for the purpose of rapidly and efficiently obtaining a large number of genes from a target sample, refers to the concept that a sample, a cell, genomic DNA, an amplified DNA fragment, and a large number of small sections and/or a large number of large sections containing the same can be processed in parallel.

### [2] High-Efficiency Analysis and Acquisition of Gene Sequence

In one aspect of the present disclosure, there is provided a highly efficient method for obtaining a gene sequence of an organism such as a microorganism. For example, when obtaining microbial gene sequences from a soil sample containing microorganisms using a conventional shotgun metagenomic method, one metagenomic library is prepared from a single soil sample (for example, 1 g of soil sample), the nucleotide sequence is determined, and the gene sequence is obtained. In this case, genomic DNA fragments derived from various microorganisms exist in a mixed state, and a method is adopted in which the genomic DNA fragments are subjected to a sequencer, the nucleotide sequence is determined, and the original gene sequence is reconstructed using the obtained overlapping part of the nucleotide sequence as a clue. However, there are many cases where it is impossible to reconstruct the original gene sequence due to the enormous diversity, partial overlapping of sequences derived from different microorganisms, and the like, and it has been difficult to efficiently obtain a large number of functional structural genes.

In the highly efficient method for obtaining a gene sequence of the present disclosure, for example, in the case of employing a 384 well microplate (one well (one large section) contains, for example, 10 droplets (small sections) as a large section from a single soil sample (for example, 1 g of soil sample), 384 metagenomic libraries are prepared, these libraries are subjected to a sequencer all at once or separately as necessary to obtain a nucleotide sequence, and thus a genome sequence derived from each microbial cell contained in the soil sample can be reconstructed with high accuracy, thereby achieving efficient acquisition of a large number of functional structural genes.

That is, a step of determining a nucleotide sequence and a step of obtaining a gene by analyzing the nucleotide sequence obtained in the above step using a computer and estimating a region encoding a protein are added after the steps according to [1]-5 or [1]-6, thereby providing a highly efficient method for obtaining a microbial gene sequence.

More specifically, this step can be performed using, for example, a next generation sequencer (NGS). NGS is a term used in comparison with a first generation sequencer using the Sanger method, and there are various principles, but a large amount of nucleotide sequences can be analyzed at low cost and in a short time by an ultra-parallel processing.

That is, when sequencing nucleotide sequences using NGS, a method of fragmenting genomic DNA, sequencing the fragmented sequences, and finally connecting them to determine the sequence is common, Fragmented DNA sequences are referred to as “read”, and their lengths are referred to as “read length”. Since NGS has a different sequencing mechanism depending on a manufacturer and a model, a read length that can be sequenced, a time required for sequencing, and the like are different. In a case where sequencing is performed using a fragment of a short read such as several hundred base pairs, when there is a repetitive sequence region longer than the fragment of the short read, a large defect, or the like in the genome sequence to be analyzed, sequence accuracy may deteriorate. In this case, when an unknown gene sequence is determined, a DNA sequence fragmented for a certain length, such as 10,000 base pairs, as compared with the short read, that is, a so-called “long read” may be used.

More specifically, examples of the NGS suitable for sequencing the short read include MiniSeq, MiSeq, NextSeq, HiSeq, and HiSeq X series of Illumina, Inc., DNBSEQ series of MOI Tech, and the like. Examples of NGS suitable for sequencing the long read include PacBio RS II manufactured by Pacific Biosciences (PacBio).

In the present disclosure, NGS suitable for sequencing short reads and NGS suitable for sequencing long reads are used in combination and operated in parallel, whereby a large amount of nucleotide sequences can be analyzed at low cost in a short time.

### [3] Method for Preserving Nucleic Acids (Genomic DNA)

In one aspect of the present disclosure, there is provided a method for preserving nucleic acids (for example, genomic DNA) such as a microorganism. Soil (including seabed soil), sea water, river water, feces, sludge (including activated sludge), industrial wastewater, waste, biological samples derived from plants or animals are rich in microbial genes with diversity, and among these microbial genes, there are many industrially useful genes with unknown functions. A genetic engineering or metabolic engineering approach has been performed in which a gene encoding an enzyme or a protein constituting a metabolic pathway of a microorganism existing in nature is cloned and introduced into a host such as E. coli or yeast/coryneform bacteria which is easily genetically modified or cultured to create a microorganism producing various compounds such as chemical raw materials. In order to newly construct a desired microorganism such as highly producing a target compound or protein, it is important to obtain a gene encoding a protein expressing desired traits such as a desired enzyme activity from nature.

As a preliminary step of obtaining a large number of gene groups from nature and determining the nucleotide sequence, a method for preserving a nucleic acid (for example, genomic DNA) library derived from a large number of microorganisms may be useful. That is, in the present disclosure, there is provided a method for preserving a microbial genomic DNA, the method including a step of preserving a nucleic acid (for example, metagenomic DNA) library of the present disclosure obtained in the above [1], for example; a container (large section) accommodating gelled droplets (small sections) as it is or after adding a substance that suppresses nucleic acid (for example, DNA) degradation at room temperature or less. Here, the temperature which is equal to or lower than the room temperature may be 4 °C or lower, preferably 20 °C or lower, more preferably −80 °C or lower, and most preferably −80 °C. By allowing a substance that suppresses DNA degradation to coexist, it is possible to preserve even in a higher temperature state. Examples of the substance that suppresses DNA degradation include chelating reagents such as ethylenediaminetetraacetic acid (EDTA) and citric acid.

### (Composition/Kit)

One aspect of the present disclosure provides compositions or kits that may be used in the methods of the present disclosure. In the present invention, a composition for amplifying nucleic acids in a cell at a single cell level may be provided. The composition may contain a gel capsule or a material thereof. Using gel capsules can be advantageous for amplifying nucleic acids in cells at a single cell level, as described elsewhere herein. In the present invention, a composition for preparing a genomic library may be provided. Using gel capsules can be advantageous for preparing nucleic acid libraries (for example, the metagenome), as described elsewhere herein.

In one embodiment, a composition for amplifying nucleic acids in cells at a single cell level, containing gel capsules or materials thereof and cells at a single cell state, may be provided. The compositions may be subjected to the steps of the methods described elsewhere herein and used for nucleic acid amplification at a single cell level. In the present invention, a composition for preparing a genomic library, containing gel capsules or materials thereof and cells at a single cell state, may be provided. The compositions may be subjected to the steps of the methods described elsewhere in the specification and used to prepare genomic libraries. In the present invention, there may be provided a composition for sequencing nucleic acids in cells at a single cell level, containing gel capsules or materials thereof and cells at a single cell state. The compositions may be subjected to the steps of the methods described elsewhere in the specification and used for sequencing nucleic acids in cells at a single cell level.

In one embodiment, a composition containing lysis reagents for amplifying nucleic acids in a cell at a single cell level, is provided. The lysis reagent may contain at least one selected from the group consisting of lysozyme, labiase, yatalase, achromopeptidase, protease, nuclease, zymolyase, chitinase, lysostaphin, mutanolysin, sodium dodecyl sulfate, sodium lauryl sulfate, potassium hydroxide, sodium hydroxide, phenol, chloroform, guanidine hydrochloride, urea, 2-mercaptoethanol, dithiothreitol, TCEP-HCL, sodium cholate, sodium deoxycholate, Triton X-100, Triton X-114, NP-40; Brij-35, Brij-58, Tween 20, Tween 80, octyl glucoside, octyl thioglucoside, CHAPS, CHAPSO, dodecyl-β-D-maltoside, Nonidet P-40, and Zwittergent 3-12.

In one embodiment, a kit for amplifying nucleic acids in a cell at a single cell level, may be provided. The kit may contain, for example, the material of the gel capsule and one or more reagents as necessary. Examples of the one or more reagents include a lysis reagent.

### (Data, Database, Data Structure, Data Processing, and the Like)

In the present disclosure, it is possible to select one to be subjected to data acquisition or analysis from the collection of libraries of the present disclosure. For example, in the present disclosure, a subpopulation containing cells or cell-like structures may be generated by selecting from a collection of cells or cell-like structures based on the nucleic acid sequences of the cells and cell-like structures. This process may include a step of generating the subpopulation containing at least one cell or cell-like structure. The generation of subpopulations can reduce the labor of steps such as sequencing and the creation of genomic drafts based on sequencing reads.

In one embodiment of the present disclosure, separately provided cells or cell-like structures can be sorted based on nucleic acid information derived from the cells or cell-like structures. As necessary, it is possible to analyze the sorted cells or cell-like structures. Regarding the sorting, sorting can be performed in several manners, for example, sequencing from PCR, sequencing partial sequences, checking for the presence or absence of specific gene sequences, and referring to DNA yield.

In one embodiment of the present disclosure, after sequencing, nucleic acid information derived from cells or cell-like structures may be sorted. After nucleic acid information derived from cells or cell-like structures is provided as a collection of nucleic acid information for each cell or cell-like structure, the nucleic acid information can be sorted for each cell or cell-like structure based on all or a part of the nucleic acid information. As necessary, the sorted nucleic acid information can be analyzed.

In one embodiment of the present disclosure, the obtained sequence information can be recorded as a database. The database may be recorded on an automatic data construction and provision system. The database can store sequence information derived from one cell or cell-like structure separately. Each sequence information can be classified and grouped. As the classification, it is desirable to classify the biological species. The classified clusters do not have contamination of sequence information of other species organisms, and complete sequence information in the clusters can be constructed based on the contamination. During construction of the complete sequence information, reclassification can also be performed. The information obtained by the analysis can also be used for refining the classification of sequence information derived from one newly obtained cell or cell-like structure.

In one aspect, the present disclosure provides a method for obtaining a nucleic acid sequences encoding genes in cells or cell-like structures and/or amino acid sequences encoded by the nucleic acid sequences, in another expression, a method for obtaining a “gene (sequence)”. The obtaining method includes: a step of providing two or more cells or cell-like structures; a step of generating a small section containing one or more cells or cell-like structures, from the cells or cell-like structures; a step of lysing the cells or cell-like structures contained in the small section such that the nucleic acids in the cells or cell-like structures are eluted and retained in the small section; a step of bringing the nucleic acid into contact with a nucleic acid amplification reagent to amplify the nucleic acids in the small section to obtain an amplification product, as necessary; a step of accommodating one or a plurality of the large sections including the small sections containing the amplification product, in a container for nucleotide sequencing, as necessary; a step of determining a nucleotide sequence of the amplification product, a step of analyzing the nucleotide sequence obtained in the above step; and a step of analyzing a region encoding genes in the nucleotide sequence to obtain a nucleic acid sequence encoding genes and/or an amino acid sequence encoded by the nucleic acid sequence. It is understood that any combination of any embodiments elsewhere herein may be applied to each step in the obtaining methods of the present disclosure.

In another aspect, the present disclosure provides a method for creating a database of sequences encoding genes in cells or cell-like structures, the method including: a step of providing two or more cells or cell-like structures; a step of generating a small section containing one or more cells or cell-like structures, from the cells or cell-like structures; a step of lysing the cells or cell-like structures contained in the small section such that the nucleic acids in the cells or cell-like structures are eluted and retained in the small section; a step of bringing the nucleic acid into contact with a nucleic acid amplification reagent to amplify the nucleic acids in the small section to obtain an amplification product, as necessary; a step of accommodating one or a plurality of the large sections including the small sections containing the amplification product, in a container for nucleotide sequencing, as necessary; a step of determining a nucleotide sequence of the amplification product; a step of analyzing the nucleotide sequence obtained in the above step; a step of analyzing a region encoding genes in the nucleotide sequence to obtain a nucleic acid sequence encoding genes and/or an amino acid sequence encoded by the nucleic acid sequence; and a step of creating a database of sequences encoding genes by using the nucleic acid sequence encoding genes and/or the amino acid sequence encoded by the nucleic acid sequence, and as necessary, information on cells or cell-like structures. It is understood that any combination of any embodiments elsewhere herein may be applied to each step in the database creation method of the present disclosure.

In another aspect, the present disclosure provides a method for generating a data structure that constitutes a sequence encoding genes in cells or cell-like structures, the method including: a step of providing two or more cells or cell-like structures; a step of generating a small section containing one or more cells or cell-like structures, from the cells or cell-like structures; a step of lysing the cells or cell-like structures contained in the small section such that the nucleic acids in the cells or cell-like structures are eluted and retained in the small section; a step of bringing the nucleic acid into contact with a nucleic acid amplification reagent to amplify the nucleic acids in the small section to obtain an amplification product, as necessary, a step of accommodating one or a plurality of the large sections including the small sections containing the amplification product, in a container for nucleotide sequencing, as necessary; a step of determining a nucleotide sequence of the amplification product; a step of analyzing the nucleotide sequence obtained in the above step; a step of analyzing a region encoding a gene in the nucleotide sequence to obtain a nucleic acid sequence encoding genes and/or an amino acid sequence encoded by the nucleic acid sequence; and a step of generating a data structure of sequences encoding genes, which are defined by the nucleic acid sequence encoding genes and/or the amino acid sequence encoded by the nucleic acid sequence, and as necessary, information on cells or cell-like structures. It is understood that any combination of any embodiments elsewhere As used herein may be applied to each step in the data structure creation method of the present disclosure.

In another aspect, the present disclosure provides a database generated by the methods of the present disclosure.

In another aspect, the present disclosure provides a data structure generated by the methods of the present disclosure.

In one embodiment, the database and data structure of the present disclosure include information indicating that the database and data structure were generated by the generation method of the present disclosure. Such information includes, but is not limited to, sequence information, information on a large section used (specifically, a barcode sequence (a short sequence represented by about 10 bases)), a gene name (predicted from an amino acid sequence), estimated origin biological information, and the like.

In a preferred embodiment, the database and the data structure of the present disclosure include full-length gene sequence percentages. The full-length gene sequence percentage can determine whether the quality of the contained nucleic acid sequence or amino acid sequence can be determined, and the present disclosure can easily evaluate the quality of data contained in the database and the data structure by having such fields.

In one embodiment, the database is provided such that the full-length gene sequence percentage of nucleic acid sequences and/or amino acid sequences encoding the contained genes is 10% or more, 15% or more, 20% or more, 25% or more, 30% or more, 35% or more, 40% or more, 45% or more, or 50% or more. A nucleic acid library having such a high full-length gene sequence percentage is not provided in the conventional technology, and the present disclosure can provide a library of very high quality, and can provide data of high quality with higher accuracy and higher coverage in subsequent nucleotide sequence analysis, amino acid sequence analysis, analysis of gene coding sequence, and the like.

In one embodiment, the database or data structure of the present disclosure includes fields related to a coding sequence, and the fields related to a coding sequence are linked to contigs used in creating a genome database or a metagenome database. In another embodiment, the fields related to the coding sequence of the present disclosure include a field that identifies whether or not the coding sequence is a complete code.

In specific embodiments, for example, the step of analyzing in a method for analyzing amino acid or nucleic acid sequences of the present disclosure includes binning a contig to generate a genomic database. As a method for binning such a contig, a known method can be used, but metaBAT2 (PeerJ 7: e7359 https://doi.org/10.7717/peerj.7359), VAMB (Nat Biotechnol 39, 555-560 (2021), https://doi.org/10.1038/s41587-020-00777-4), and SemiBin2 (Bioinformatics, Volume 39, Issue Supplement_1, June 2023, Pages 121-129, https://doi.org/10.1093/bioinformatics/btad209) are preferable.

The computer program, the database, or the data structure of the present disclosure may be stored in a storage medium. The storage medium may be a non-transitory storage medium.

References cited in the present disclosure, such as scientific literature, patents, patent applications, and the like, are hereby incorporated by reference in the present disclosure in their entirety to the same extent as each was specifically described.

The present disclosure has been described above with reference to preferred embodiments for easy understanding. Hereinafter, the present disclosure will be described based on examples, but the above description and the following examples are provided for the purpose of illustration only and not for the purpose of limiting the present disclosure. Accordingly, the scope of the present disclosure is not limited to the embodiments or examples specifically described As used herein, but is limited only by the claims.

### EXAMPLES

The embodiments described below do not limit the content of the present disclosure described in the claims. In addition, not all of the configurations described below are essential requirements of the present disclosure.

### [Example 1] Preparation of Metagenomic Libraries from Soil Samples

5 g of a soil sample as a sample was collected in a tube having a volume of 15 mL, and suspended in 6 mL of phosphate buffered saline (PBS) (Dulbecco's phosphate-buffered saline, 14190-144, Thermo Fisher Scientific Inc.). After allowing the mixture to stand for 30 minutes, the supernatant was recovered and centrifuged at 10,000×g for 5 minutes to recover the precipitate. This process was repeated twice, and the precipitate obtained by centrifugation at 8,000× g for 5 minutes was resuspended in PBS. The soil particles were precipitated by centrifugation at 300× g for 5 minutes, and the supernatant was recovered to collect soil microorganisms.

The cell concentration in the prepared cell suspension was measured with a bacteria calculator using an optical microscope, and ultra-low melting point agarose (AS030-10G, Sigma-Aldrich) was added such that the agarose concentration of the cell suspension was 1.5% to prepare a soil microorganism suspension to be used for preparing gelled droplets (final cell concentration: 2.1×10⁴ cells/μL).

Microdroplet preparation and encapsulation of single cell soil microorganism into microdroplets were performed using a Droplet Generator (On-chip Biotechnologies Co., Ltd., 60001). Specifically, a soil microorganism suspension was introduced from an inlet 1, a fluorine-based oil (On-chip Biotechnology, Inc., 008-fluoroSurfactant) (hereinafter referred to as “oil”) was introduced from an inlet 2, and the soil microorganism suspension was sheared to prepare microdroplets having a diameter of 30 μm, and the microdroplets were accumulated in an outlet, and then recovered in a tube having a volume of 0.5 mL.

The tubes were then cooled on ice for 15 minutes and the microdroplets were gelled with ultra-low melting agarose. The gelled microdroplet is a gelled droplet. Since the diameter of the microdroplet is 30 μm, the diameter of the gelled droplet is also 30 μm. Next, 100 μL of 10% 1H,1H,2H,2H-perfluoro-1-octanol (Sigma-Aldrich) was added to the tube, and the oil of the lower layer was removed. Then, acetone (FUJIFILM Wako Pure Chemical Corporation) (500 μL) and isopropanol (500 μL) (FUJIFILM Wako Pure Chemical Corporation) were added in this order and centrifugally washed to remove the oil. Further, 500 μL of PBS was added thereto, centrifugal washing was performed 3 times, and the gelled droplets were brought into a state of being suspended in an aqueous layer (PBS). Subsequently, the gelled droplets were sequentially immersed in a lysis reagent as a lysis reagent, parts other than the collection target substance such as cell walls of cells were lysed inside the gelled droplets, and genomic DNA was eluted into the gelled droplets.

Specifically, lysozyme (50 U/μL) (R1804M, Epicentre), which is one of lysis reagents, was added to the tube, and the tube was allowed to stand at 37 °C 2 hours to lyse the cells. Next, protease K (1 mg/mL) (MC5005, Promega) and sodium dodecyl sulfate (SDS) 0.5% (71736-100 ML, Sigma-Aldrich), which are one of lysis reagents, were added to the tube, and the tube was allowed to stand at 40 °C 15 hours. Thereafter, the cells were lysed, and then centrifuged and washed 5 times to remove the protease and components (contaminants) other than genomic DNA of the lysed cells from the tube. Subsequently, the gelled droplets were immersed in Buffer D2 (QIAGEN N.V.), which is an aqueous solution containing potassium hydroxide as one of the lysis reagents, to lyse the residual parts and denature the genomic DNA. Since the gelled droplet is immersed in the lysis reagent for a short time, the eluted genomic DNA is not flowed out of the gelled droplet by the lysis reagent and is retained in the gelled droplet.

As described above, by lysing cells with a plurality of types of lysis reagents, it is possible to collect target genomic DNA, and by performing centrifugal washing after immersion in the lysis reagent, it is possible to remove contaminants such as the lysis reagent and components other than polynucleotides of the lysed cells, and extract and purify genomic DNA without inhibiting the subsequent genomic DNA amplification reaction.

An amplification reagent was added to a tube containing gelled droplets retaining genomic DNA denatured in a potassium hydroxide solution (Buffer D2), and the gelled droplet were immersed in the amplification reagent. Specifically, a multiple displacement amplification (MDA) method using phi29 DNA polymerase, which is a strand displacement DNA synthase, was used. Here, the whole genome amplification reaction reagent was immersed in REPLI-g Single Cell Kit (QIAGEN N.V.), and a whole genome amplification reaction was performed for 3 hours (S1000 Thermal cycler, Bio-Rad Laboratories, Inc.). The amplification reagent (REPLI-g Single Cell Kit) contains a component that neutralizes a potassium hydroxide solution (Buffer D2).

The gelled droplets after the whole genome amplification were centrifugally washed using PBS, and then stained with a fluorescent DNA intercalator SYBR green (5760 A, Takara Bio Inc.) as a staining reagent. For staining, other known staining reagents such as EvaGreen (31000, Cosmo Bio Co., Ltd.) may be used.

The metagenomic library of the present disclosure could be prepared by sorting gelled droplets retaining genomic DNA amplified to a predetermined level or more by a flow cytometer (BD FACSMelody Cell Sorter, BD Biosciences) and individually recovering 10 gelled droplets on a plate (HSP3801, Bio-Rad Laboratories, Inc.) as a storage container.

### [Example 2] Acquisition of Microbial Genes from Metagenomic Libraries

Library preparation by QIAseq FX DNA Library kit (QIAGEN N.V., 180475) was performed on the dispensed samples, and 2×150 bp paired end reads (120 Gb) were obtained by sequencing using DNBSEQ G400 (MGI Tech). The type of sequencing is not limited as long as sequencing can be performed under the same conditions.

Sequence data (read sequence) was assembled using SPAdes (Journal of Computational Biology, 19 (5): 455-477 (2012) to obtain a contig, and then gene estimation on the contig was performed using annotation software Prokka (Bioinformatics, 30 (14); 2068-2069 (2014)).

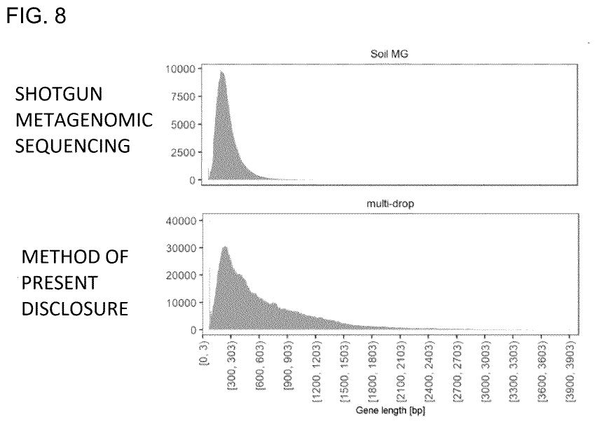

As a result, 5691,207 genes were obtained from the analyzed soil samples. Among them, the number of obtained genes having a length of 200 amino acid residues or more was 2,356,273 (Table 1). As a method for obtaining genes from a soil sample, a shotgun metagenomic sequencing method (Nature Biotechnology 35:833-844 (2017)) is known. In this method, DNA extracted from a soil sample is sequenced. As a result of performing the shotgun metagenomic sequencing method on the same sample, the total number of genes was 698,652, and among them, the number of obtained genes having a length of 200 amino acid residues or more was 20,498. Both indexes demonstrated that the method described in the present disclosure outperformed the conventional approach (Table 1). In addition, when comparing the distribution of the lengths of the genes obtained from the soil samples, it was shown that the method according to the present disclosure exhibits a distribution biased toward longer gene sequences compared to the shotgun metagenomic sequencing method (FIG. 8).

TABLE 1
Number of gene sequences obtained from soil samples

| | | Number of genes obtained having |
|---|---|---|
| | Total number of | length of 200 amino acid residues |
| Method | genes obtained | or more |
| Shotgun metagenomic sequencing | 698,652 | 20,498 |
| Method of present invention | 5,691,207 | 2,356,273 |

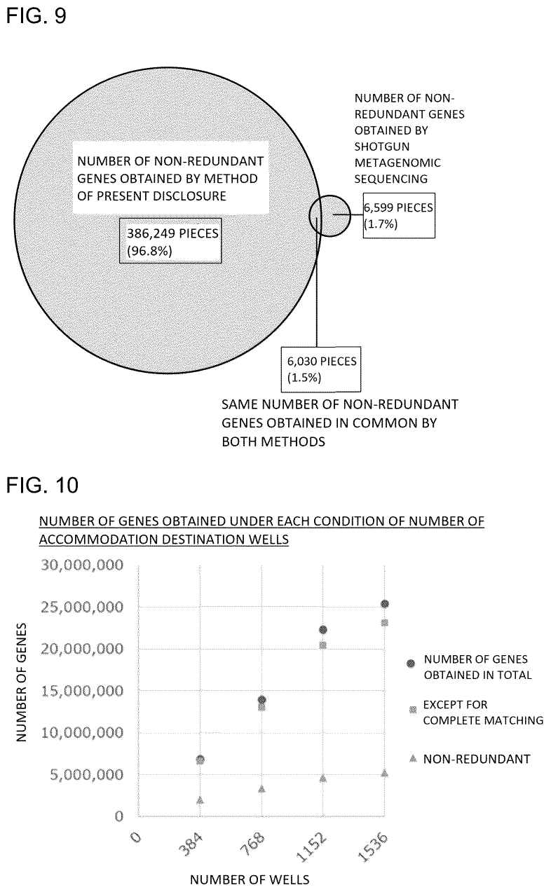

In addition, when comparing the distribution of the lengths of the genes obtained from the same soil samples, it was shown that the method according to the present disclosure exhibits a distribution biased toward longer gene sequences compared to the shotgun metagenomic sequencing method (FIG. 8). Next, among the genes having a length of 200 amino acid residues or more obtained by each method, the commonality of gene sequences (hereinafter referred to as a “non-redundant gene”) obtained by excluding genes of which sequences coincide with a predetermined level or more was analyzed. As a result, when the total of the number of non-redundant genes obtained only by the method of the present disclosure, the number of non-redundant genes obtained only by the shotgun metagenomic sequencing method, and the number of non-redundant genes obtained in common by both methods, that is, 398,878, is taken as 100%, the number of non-redundant genes obtained in common by both methods is 6,030, which is 1.5% of the total, and the number of non-redundant genes obtained only by the shotgun metagenomic sequencing method is 6,599, which is 1.7% of the total. On the other hand, the number of non-redundant genes obtained only by the method of the present disclosure is 386,249, which is 96.8% of the total, and by using the method according to the present disclosure, it was confirmed that it was possible to acquire a large number of genes that could not be obtained by shotgun metagenomic analysis (FIG. 9).

Further, for another soil sample, 10 gelled droplets obtained by performing the same treatment were individually recovered per well in a microplate (HSP3801, Bio-Rad Laboratories, Inc.), and the same analysis was performed. At this time, the test was performed under each condition in which the number of accommodation destination wells was 384 wells (for 1 plate), 768 wells (for 2 plates), 1,152 wells (for 3 plates), and 1,536 wells (for 4 plates). As a result, the total number of genes obtained was 6,842,892, 13,921,293, 22,298,612, and 25,383,609, respectively (FIG. 10). In addition, the number of genes excluding the perfect matching sequence was also 6,567,645, 13,024,107, 20,425,488, and 23,066,293, respectively. The number of non-redundant genes was also 1,967,477, 3,318,317, 4,583,402, and 5,186,422, respectively, and it was shown that gene information can be recovered in a diverse and large-scale manner from one sample by increasing the number of accommodation destination wells.

### [Example 3] Acquisition of Genome Information by Single Drop Method

A one-cell amplified genomic library was prepared from human intestinal microorganisms collected from human feces as a sample. The experiment was conducted by collecting human feces into a 1.5 mL tube (1212-10, SSIbio) and homogenizing it using a disruption tool in 500 μL of phosphate-buffered saline (PBS) (Dulbecco's phosphate-buffered saline, 14190-144, Thermo Fisher Scientific Inc.) until no solid material remained. The operation of centrifuging at 1,500×g for 30 seconds and recovering the supernatant was repeated 2 times, and then centrifuging at 8,000×g for 5 minutes to collect the mouse intestinal microorganisms.

After centrifuging the bacterial pellet twice with PBS for washing, the bacterial pellet was suspended in PBS to obtain a cell suspension of human intestinal microorganism. The cell concentration in the prepared cell suspension was measured, and ultra-low melting point agarose (A5030-10G, Sigma-Aldrich) was added to have a final concentration of 1.5%, thereby preparing an intestinal microorganism suspension to be used for preparation of a gel capsule (final cell concentration: 7×10³ cells/μL), Microdroplet preparation and encapsulation of single cell human intestinal microorganism into microdroplets were performed using a Droplet Generator (On-chip Biotechnologies Co., Ltd., 60001). Specifically, an intestinal microorganism suspension was introduced from the inlet 1, a fluorine-based oil (On-chip Biotechnology, Inc., 008-fluoroSurfactant) (hereinafter referred to as “oil”) was introduced from the inlet 2, and the human intestinal microorganism suspension was sheared to prepare microdroplets having a diameter of 30 μm, and the microdroplets were accumulated in an outlet, and then recovered in a tube having a volume of 1.5 mL.

The tubes were then cooled on ice for 15 minutes and the microdroplets were gelled with ultra-low melting agarose. 100 μL of 1H,1H,2H,2H-perfluoro-1-octanol (Sigma-Aldrich) was added to the tube, and the oil of the lower layer was removed. Then, acetone (FUJIFILM Wako Pure Chemical Corporation) (500 μL) and isopropanol (500 μL) (FUJIFILM Wako Pure Chemical Corporation) were added in this order and centrifugally washed to remove the oil. Further, 500 μL of PBS was added thereto, centrifugal washing was performed 3 times, and the gel capsules were brought into a state of being suspended in an aqueous layer (PBS).

Subsequently, the gel capsules were sequentially immersed in a lysis reagent, parts other than the collection target substance such as cell walls of cells were lysed inside the gel capsules, and genomic DNA was eluted into the gel capsules. Specifically, lysozyme (10 U/μL) (R1804M, Epicentre), which is one of lysis reagents, was added to the tube to lyse the cells. Next, protease K (1 mg/mL) (MC5005, Promega) and sodium dodecyl sulfate (SDS) 0.5% (71736-100 ML, Sigma-Aldrich), which are one of lysis reagents, were added to the tube. Thereafter, the cells were lysed, and then centrifuged and washed 5 times to remove the protease and components (contaminants) other than genomic DNA of the lysed cells from the tube. Subsequently, the gel capsules were immersed in Buffer D2 (QIAGEN N.V.), which is an aqueous solution containing potassium hydroxide as one of the lysis reagents, to lyse the residual parts and denature the genomic DNA. As described above, by lysing cells with a plurality of types of lysis reagents, it is possible to collect target genomic DNA, and by performing centrifugal washing after immersion in the lysis reagent, it is possible to remove contaminants such as the lysis reagent and components other than polynucleotides of the lysed cells, and purify genomic DNA without inhibiting the subsequent genomic DNA amplification reaction.

An amplification reagent was added to a tube containing gel capsules retaining genomic DNA denatured in a potassium hydroxide solution (Buffer D2), and the gel capsules were immersed in the amplification reagent. Specifically, a multiple displacement amplification (MDA) method using phi29 DNA polymerase, which is a strand displacement DNA synthase, was used. Here, the whole genome amplification reaction reagent was immersed in REPLI-g Single Cell Kit (QIAGEN N.V.), and a whole genome amplification reaction was performed for 3 hours (S1000 Thermal cycler, Bio-Rad Laboratories, Inc.). The amplification reagent (REPLI-g Single Cell Kit) contains a component that neutralizes a potassium hydroxide solution (Buffer D2). The gel capsules after whole genome amplification were centrifuged and washed with PBS, and then stained with a fluorescent DNA intercalator using SYBR green (S7563, Thermo Fisher Scientific Inc.) as a staining reagent, as shown in FIG. 5.

Gel capsules retaining genomic DNA amplified to a predetermined level or more by a flow cytometer (BD FACSMelody Cell Sorter, BD Biosciences) were sorted, and individually recovered in a 384-well multiwell plate as a storage container. Subsequently, a library was prepared by QIAseq FX DNA Library kit (QIAGEN N.V.), and 2×150 bp paired end reads (44.1 Gb) were obtained by whole genome sequencing using NextSeq 2000 (Illumina, Inc.).

Sequence data (read sequence) was assembled using SPAdes (Bankevich et al. Journal of computational biology, 19 (5), 455-477. 2012 (http://doi.org/10.1089/cmb.2012.0021) to obtain contigs, and then the contigs were evaluated using QUAST (Gurevich et al. Bioinformatics. 2013 29 (8): 1072-5. doi: 10.1093/bioinformatics/bt: 086.). CheckM (Parks et al., Genome Research 2015, 25:1043-1055, doi: 10.1101/gr. 186072.114) was used for the evaluation of the genome completeness rate and the contamination degree.

As a result, in the total of 384 single cell genomes analyzed, the genome completeness rate exceeded 50% in 175 genomes, and exceeded 90% in 18 genomes among them. The contamination degree was as low as 6.3% on average. As a result of comparing the above 175 genomes with the international standard “Minimum information about a single amplified genome (MISAG)” (Bowers et al., Nature Biotechnology 2017 35 (8): 725-731. doi: 10.1038/nbt. 3893.4), genome information derived from 1 cell obtained from human feces was genome information evaluated as having medium to high quality.

### [Example 4] Comparison Between Single Drop Method and Multi-Drop Method

A comparative analysis was performed between a single cell genome sequencing method in which gelled droplets obtained by performing the same treatment on the same soil sample were individually recovered into wells of a plate (HSP3801, Bio-Rad Laboratories, Inc.) as a storage container at one droplet per well, the method described in the present disclosure in which 10 droplets per well were individually recovered. The results were then compared. At this time, the single cell genome sequencing method was carried out under the condition where the number of accommodation destination wells was set to 3,072 wells (8 plates, total of 3,072 droplets), and the present disclosure was carried out under the condition where the number of accommodation destination wells was set to 384 wells (1 plate, total 3,840 droplets). As a result, the obtained gene variation numbers were 5,708,085 and 6,019,181, respectively (Table 2). The number of obtained gene variations per 3,000 droplets to be analyzed was 5,574,302 and 4,702,485, respectively, and the present disclosure showed 0.84 times the number of obtained genes (effect) as compared with the single cell genome sequencing method. On the other hand, the analysis cost required for the present disclosure was 0.27 times higher than for the single cell genome sequencing method. Accordingly, cost effectiveness (relative ratio) was calculated as 3.07 for the method described in the present disclosure, compared to 1.00 for the single cell genome sequence method. From the above, it has been shown that the method according to the present disclosure can recover gene information from one sample in a diverse and large-scale manner, and is a significantly economically advantageous method for that purpose.

TABLE 2

| | Effect | Effect* 3,000 droplets | | | |
|---|---|---|---|---|---|
| | (Obtained gene | (Obtained gene | Effect | Cost | Cost effectiveness |
| Method | variation number) | variation number) | (Relative ratio) | (Relative ratio) | (Relative ratio) |
| Single cell genome sequencing | 5,708,085 | 5,574,302 | 1.00 | 1.00 | 1.00 |
| method (Analysis of 3,072 droplets) | | | | | |
| Present invention | 6,019,181 | 4,702,485 | 0.84 | 0.27 | 3.07 |
| (Analysis of 3,840 droplets) | | | | | |

From the above, it was shown that the method according to the present disclosure is a method capable of highly efficiently obtaining gene information from a soil sample.

### Example 5

Since soil contains many uncultured species, the metagenomic method is used for genome analysis for the purpose of collecting industrially useful microbial genetic resources and functional analysis of environmental microorganisms. However, since significantly many types of microbial DNA are mixed in the soil, it is difficult to distinguish the sequence derived from each microorganism and reconstruct a draft genome (metagenome-assembled genome (MAG)).

In this example, a new method was developed that enables large-scale collection of draft genomes by improving the efficiency of discrimination of a wide variety of microorganism sequence data in soil.

That is, by the same method as in Example 1, the metagenomic library of the present invention was created by preparing gelled droplets retaining an amplification product of soil microbial genomic DNA, sorting gelled droplets retaining genomic DNA amplified to a predetermined level or more by a flow cytometer (BD FACSMelody Cell Sorter, BD Biosciences), and individually recovering 10 gelled droplets on a 384-well plate (HSP3801, Bio-Rad Laboratories, Inc.) as a storage container. In this manner, a metagenomic library containing 10 droplets (small sections referred to in the claims) in 1 well (large sections referred to in the claims) was obtained. Library preparation by QIAseq FX DNA Library kit (QIAGEN N.V., 180475) was performed on the dispensed samples, and 2× 150 bp paired end reads (120 Gb) were obtained by sequencing using DNBSEQ G400 (MGI Tech). Sequence data (read sequence) was assembled using SPAdes (Journal of Computational Biology, 19 (5): 455-477 (2012)) to obtain contigs, then binning was performed using metagenomic binning tools metaBAT2 (PeerJ 7: e7359 https://doi.org/10.7717/peerj.7359), VAMB (Nat Biotechnol 39; 555-560 (2021). https://doi.org/10.1038/s41587-020-00777-4), and SemiBin2 (Bioinformatics, Volume 39, Issue Supplement_1, June 2023, Pages i21 i29, https://doi.org/10.1093/bioinformatics/btad209), and the obtained bins were integrated using MAGScoT (Bioinformatics, Volume 38, Issue 24, 15 Dec. 2022, Pages 5430-5433, https://doi.org/10.1093/bioinformatics/btac694) to construct MAGs derived from each sample. The sample of soil 6 was analyzed in a similar manner. QUAST (Bioinformatics. 2013 29(8): 1072-5. doi: 10.1093/bioinformatics/btt086.) and CheckM (Genome Research 2015. 25:1043-1055, doi: 10.1101/gr. 186072.114) were used to evaluate the quality of the obtained MAG. GTDB-Tk (Bioinformatics, Volume 36, Issue 6, March 2020, Pages 1925-1927, https://doi.org/10.1093/bioinformatics/btz848) was used as annotation of biological lineages for the obtained MAG. The number of MAG species was estimated by clustering with Dashing 2 (Genome Res. 2023 July; 33 (7): 1218-1227. doi: 10.1101/gr. 277655.123.).

### (Results)

As a result of analyzing the sample of soil 6 by this method, 9,086 MAGs having a genome quality standard of medium-quality or higher were constructed. The biological lineages to which these belong spanned 51 phyla, and MAGs corresponding to 5,608 species at the species level were obtained. Among them, in the sample of soil 1, 17 types of MAG were obtained by the shotgun metagenomic method, while 1,250 types of MAG were obtained by the present method. From the above, it was suggested that the present method was a method capable of efficiently collecting various types of microbial genomes from the sample of soil 1. Utilization of the present method is expected to contribute to expansion of useful microbial genetic resources and comprehensive analysis of microorganism functions in environmental samples.

### Example 6

For a certain soil sample, the cumulative relative frequency using the gene length (number of bases) as an index was confirmed for the gene population obtained by the conventional shotgun metagenomic method or the method of the present disclosure.

Specifically, sequence data was obtained from the sample of soil 1 in the same manner as in Examples 1 and 2, sequence data (read sequence) was assembled using SPAdes (Journal of Computational Biology, 19 (3): 455-477 (2012) to obtain a contig, and then gene estimation on the contig was performed using annotation software Prokka (Bioinformatics, 30 (14): 2068-2069 (2014)).

The gene estimated on the contig, that is, the coding region was evaluated by using the length and number as an analysis target. This is specifically as follows. (1) The coding regions that are presumed to encode genes in the obtained contig sequences were identified: (2) a large number of identified coding regions and the number of bases thereof were listed; and (3) the percentage was calculated by using the number of coding regions found in (2) above that were 1,000 bp or more (corresponding to approximately 333 amino acid residues) as the denominator, and the number of all coding regions as the numerator.

### (Results)

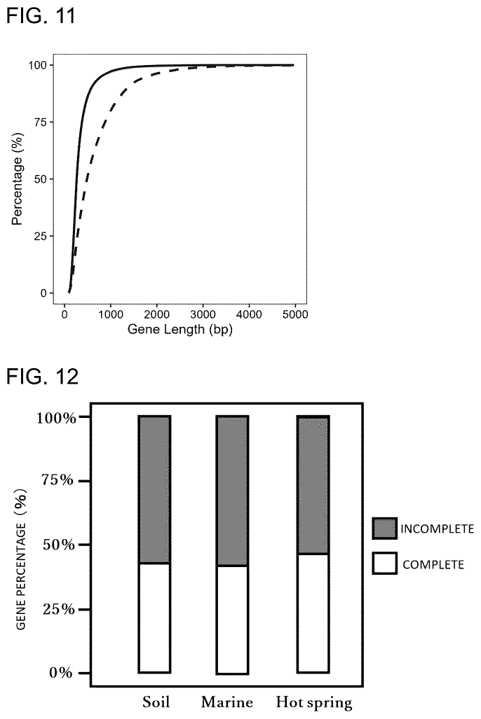

As a result, the percentage of genes having a length of 1,000 bp or less reached 97.1% by the shotgun metagenomic method (FIG. 11, solid line). On the other hand, the percentage was 80.0% in the method of the present disclosure (multi-drop method) (FIG. 11, dotted line).

That is, 20% of genes having a length of 1,000 bp (corresponding to about 333 amino acid residues) or more were contained in the method of the present disclosure (multi-drop method), which was 6 times or more as compared with the shotgun metagenomic method (2.9%). From the above, it was suggested that this method is a method capable of recovering long genes and constructing a microbial gene database with higher accuracy as compared with shotgun metagenomic analysis.

In addition, it was found that the full-length gene sequence percentage of the library obtained by the present disclosure was dramatically increased as compared with the conventional technology, and it was understood that the library provided a novel data structure or library:

### Example 7: Full-Length Gene Sequence Percentage

For various environmental samples (soil, seawater, hot spring water), success of failure of acquisition at the gene full length was determined for the gene population obtained by the method of the present disclosure.

Specifically, sequence data was obtained from the sample of soil 6, the sample of seawater 1, the sample of hot spring 3 in the same manner as in Examples 1 and 2, sequence data (read sequence) was assembled using SPAdes (Journal of Computational Biology, 19(5): 455-477 (2012) to obtain a contig, and then gene estimation on the contig was performed using annotation software Prokka (Bioinformatics, 30 (14): 2068-2069 (2014)).

The coding region estimated on the contig was evaluated as an analysis target. This is specifically as follows. (1) The coding regions that are presumed to encode genes in the obtained contig sequences were identified; (2) a large number of identified coding regions was listed; and (3) the full-length gene sequence percentage and the full-length gene sequence content percentage were calculated by using the total number of coding regions found in (2) above as the denominator, and the number of coding regions that fully fit within a contig, corresponding to full-length genes as the numerator.

### (Results)

The results are shown in FIG. 12. In FIG. 12, a case where the full length of the coding region (CDS) of a gene is contained within a contig is labeled as “complete”, and a case where the full length of the coding sequence (CDS) of a gene is not contained within a contig is labeled as “incomplete”, “Complete/complete+incomplete” can be displayed as the “full-length gene sequence percentage”. This numerical value is the full-length gene sequence content percentage with respect to the original nucleic acid library. As a result, the environmental sample (soil, hot spring, sea water) exceeded 42.3% at the minimum in this example (FIG. 12). In the conventional technology, such as shotgun metagenomic analysis, has not been evaluated on a value basis, such as full-length gene sequence percentage, which is much lower than when using the technology of the present disclosure. In the present disclosure, by introducing the full-length gene sequence percentage or the full-length gene sequence content percentage, the quality of a database, a data structure, or the like, and a library can be controlled in another aspect. In the present example, it was found that a full-length gene could be obtained at a larger proportion, and it was suggested that the obtained library, data structure, and database were not conventional, and a method capable of constructing a genome or metagenome database with a higher coverage and high accuracy was possible.

### NOTE

As described above, the present disclosure has been illustrated using the preferred embodiments of the present disclosure, but the present disclosure should not be construed as being limited to the embodiments. It is understood that the scope of the present disclosure should be interpreted only by the claims. It is understood from the description of the specific preferred embodiments of the present disclosure that those skilled in the art can implement equivalent ranges based on the description of the present disclosure and the common general knowledge. It is understood that the patents, patent applications and documents cited herein are to be incorporated by reference in their content to the present specification, as the content thereof is specifically described herein. This application claims priority to Japanese Patent Application No. 2022-160265 (filed on Oct. 4, 2022), the entire contents of which are incorporated herein by reference.

### INDUSTRIAL APPLICABILITY

The present disclosure can be utilized in the discovery of novel industrial enzymes that can be used in a wide range of fields such as the food industry, the chemical industry, the environmental industry, the pharmaceutical industry, and the medical field.

1. A nucleic acid library containing one or more nucleic acid molecules in one small section and a large section containing one or a plurality of the small sections.

2. The nucleic acid library according to claim 1, wherein the large section contains two or more small sections.

3. The nucleic acid library according to claim 1 or 2, wherein the nucleic acid molecules contained in one small section include one or more nucleic acid molecules derived from a cell or cell-like structure and/or an amplification product obtained by amplification using the nucleic acid molecule as a template.

4. The nucleic acid library according to any one of claims 1 to 3, wherein the nucleic acid library includes a library of metagenomes.

5. The nucleic acid library according to any one of claims 1 to 4, wherein the nucleic acid or amplification product includes genomic DNA derived from a cell or a cell-like structure and/or an amplification product obtained by amplification using the genomic DNA as a template.

6. The nucleic acid library according to any one of claims 1 to 5, wherein the large section contains the 5 to 15 small sections.

7. The nucleic acid library according to any one of claims 1 to 6, wherein the one or more cells or cell-like structures include two or more types of cells or cell-like structures.

8. The nucleic acid library according to any one of claims 1 to 7, wherein the small section is a gelled droplet, a droplet, or a coated droplet.

9. The nucleic acid library according to any one of claims 1 to 8, wherein the gelled droplets are gelled with any gelling material selected from the group consisting of agarose, acrylamide, photocurable resin, polyethylene glycol, gelatin, sodium alginate, Matrigel, collagen, and hydrogel.

10. The nucleic acid library according to any one of claims 1 to 9, wherein, in the amplification, amplification is performed while maintaining a gel state within a gel capsule.

11. The nucleic acid library according to any one of claims 1 to 10, wherein the gelled droplet has a diameter of about 1 to 250 μm.

12. The nucleic acid library according to any one of claims 1 to 11, wherein the large section is a well forming portion of a microplate or a microtube.

13. A combination of nucleic acid libraries, comprising a plurality of nucleic acid libraries according to any of claims 1 to 12.

14. The nucleic acid library or the combination of nucleic acid libraries according to any one of claims 1 to 13, wherein the nucleic acids contained in the library include a barcode sequence unique to the large section.

15. The nucleic acid library or the combination of nucleic acid libraries according to any one of claims 1 to 14, wherein the nucleic acids contained in the library contained in the small section include nucleic acids amplified to a predetermined length or more.

16. The nucleic acid library or the combination of nucleic acid libraries according to any one of claims 1 to 15, wherein, in the library contained in the small section, a proportion of coding regions on contigs obtained from read sequences of the nucleic acids that are 1,000 bp or more is 5% or more.

17. The nucleic acid library or the combination of nucleic acid libraries according to any one of claims 1 to 16, wherein, in the library contained in the small section, a full-length gene sequence content percentage in the coding regions on the contig obtained from the read sequences of the nucleic acids is 20% or more.

18. The nucleic acid library or the combination of nucleic acid libraries according to any one of claims 1 to 17, wherein, in the library contained in the small section, a full-length gene sequence content percentage in the coding regions on the contigs obtained from the read sequences of the nucleic acids is 30% or more.

19. A method for preparing a nucleic acid library, comprising:
a step of providing two or more cells or cell-like structures;
a step of generating a small section containing one or more cells or cell-like Structures, from the cells or cell-like structures;
a step of lysing the cells or cell-like structures contained in the small section such that the nucleic acids in the cells or cell-like structures are eluted and retained within the small section;
a step of bringing the nucleic acids into contact with a nucleic acid amplification reagent to amplify the nucleic acids within the small section and obtain an amplification product, as necessary; and
a step of accommodating one or a plurality of the large sections containing the small sections containing the amplification product, in a container for nucleotide sequencing, as necessary.

20. The method according to claim 19, wherein the large sections are all accommodated in the container.

21. The method according to claim 19 or 20, wherein the cells or cell-like structures are provided in a form of a suspension.

22. The method according to any one of claims 19 to 21, wherein during the amplification, after cells or cell-like structures contained in the small section are lysed, nucleic acids containing the genome in the cells or cell-like structures are eluted into the small section and retained in the small section.

23. The method according to any one of claims 19 to 22, wherein the step of obtaining the amplification product includes adding a barcode sequence unique to the large section to the nucleic acid.

24. The method according to any one of claims 19 to 23, wherein the step of accommodating the small sections in the large sections includes removing nucleic acids other than nucleic acids amplified to a predetermined length or more.

25. The method according to any one of claims 19 to 24, wherein the sample containing two or more cells or cell-like structures is a single sample.

26. The method according to any one of claims 19 to 25, wherein the sample containing two or more cells or cell-like structures include one or more selected from the group consisting of soil (including seabed soil), seawater, river water, lake water, feces, saliva, skin, sputum, sludge (including activated sludge), industrial wastewater, tissues derived from animals and plants, and surgical irrigation solution.

27. The method according to any one of claims 19 to 26, wherein the number of small sections accommodated in one large section is 2 or more.

28. The method according to any one of claims 19 to 27, wherein the number of small sections accommodated in one large section is 5 to 15.

29. The method according to any one of claims 19 to 28, wherein the cells or cell-like structures include two or more types.

30. A method for preparing a metagenomic library according to any one of claims 19 to 29, wherein one or more or one or more types are 2 to 3 or 2 to 3 types.

31. The method according to any one of claims 19 to 30, wherein the small section is a gelled droplet, a droplet, or a coated droplet.

32. The method according to any one of claims 19 to 31, wherein the gelled droplets are gelled with any gelling material selected from the group consisting of agarose, acrylamide, photocurable resin, polyethylene glycol, gelatin, sodium alginate, Matrigel, collagen, and hydrogel.

33. The method according to any one of claims 19 to 32, wherein the gelled droplet has a diameter of about 1 to 250 μm.

34. The method according to any one of claims 19 to 33, wherein the step of generating the small sections is performed by previously containing a component having a gelling ability in the cell or cell-like structure suspension, then flowing the cell or cell-like structure suspension having a gelling ability into a microchannel, and shearing the suspension with oil.

35. The method according to any one of claims 19 to 34, wherein the large section is a well of a microplate or a microtube.

36. A method for analyzing nucleic acid sequences in cells or cell-like structures, the method comprising:
a step of providing two or more cells or cell-like structures;
a step of generating a small section containing one or more cells or cell-like structures, from the cells or cell-like structures;
a step of lysing the cells or the cell-like structures contained in the small section such that the nucleic acids in the cells or the cell-like structures are eluted and retained in the small section;
a step of obtaining an amplification product by bringing the nucleic acid into contact with the nucleic acid amplification reagent and amplifying the nucleic acid in the small section;
a step of accommodating one or a plurality of the large sections including the small sections containing the amplification product, in a container for nucleotide sequencing, as necessary;
a step of determining a nucleotide sequence of the amplification product;
a step of analyzing the nucleotide sequence obtained in the step;
a step of analyzing the amino acid sequence encoded by the nucleotide sequence, as necessary; and
a step of analyzing a region encoding a gene, as necessary.

37. The method according to claim 36, wherein the large sections are all accommodated in the container.

38. The analysis method according to claim 36 or 37, wherein the step of obtaining the amplification product includes adding a barcode sequence unique to the large section to the nucleic acid.

39. The analysis method according to any one of claims 36 to 38, comprising combining small sections containing the amplification product into a smaller number of sections.

40. The analysis method according to any one of claims 36 to 38, comprising combining small sections containing the amplification product into one section.

41. The analysis method according to any one of claims 36 to 40, further comprising sorting only nucleic acids amplified to a predetermined length or more for the amplification product.

42. The analysis method according to any one of claims 36 to 41, wherein the step of analyzing the region encoding the genes includes calculating a full-length gene sequence percentage.

43. A method for analyzing a metagenome, comprising:
a step of providing two or more cells or cell-like structures that constitute the metagenome;
a step of generating a small section containing one or more cells or cell-like structures, from the cells or cell-like structures;
a step of lysing the cells or the cell-like structures contained in the small section such that the nucleic acids in the cells or the cell-like structures are eluted and retained in the small section;
a step of obtaining an amplification product by bringing the nucleic acid into contact with the nucleic acid amplification reagent and amplifying the nucleic acid in the small section;
a step of accommodating one or a plurality of the large sections including the small section containing the amplification product, in a container for nucleotide sequencing, as necessary;
a step of determining a nucleotide sequence of the amplification product in the small section;
a step of analyzing the nucleotide sequence obtained in the step to obtain nucleic acid sequence information of the metagenome;
a step of analyzing the amino acid sequence encoded by the nucleotide sequence, as necessary; and
a step of analyzing a region encoding a gene, as necessary.

44. An analysis system for a nucleic acid sequence in cells or cell-like structures, the analysis system comprising:
a small section generation unit that generates a small section containing one or more cells or cell-like structures, from the cells or cell-like structures;
a nucleic acid lysis unit that lyses the cells or the cell-like structures contained in the small section such that the nucleic acids in the cells or the cell-like structures are eluted and retained in the small section;
an amplification reagent accommodation unit that accommodates a nucleic acid amplification reagent used for obtaining an amplification product by coming into contact with the nucleic acid and amplifying the nucleic acid in the small section;
an accommodation unit that accommodates one or a plurality of the large sections including the small sections containing the amplification product, as necessary;
a nucleotide sequencing unit that determines a nucleotide sequence of an amplification product in the small section;
a nucleotide sequence analysis unit that analyzes the nucleotide sequence obtained in the step;
an amino acid sequence analysis unit that analyzes an amino acid sequence encoded by the nucleotide sequence, as necessary, and
a coding region analysis unit that analyzes a region encoding a gene, as necessary.

45. The method according to any one of claims 36 to 43 or the analysis system according to claim 44, wherein the nucleotide sequences of the amplification products are determined simultaneously in parallel.

46. The method according to any one of claims 36 to 43 or 45, or the analysis system according to claim 44 or 45, wherein one or a plural number is 96 or more.

47. The method according to any one of claims 36 to 43 or 45 to 46, or the analysis system according to any one of claims 44 to 46, wherein the determination of the nucleotide sequence is performed using a next generation sequencer.

48. A method for obtaining a nucleic acid sequence encoding genes in cells or cell-like structures and/or an amino acid sequence encoded by the nucleic acid sequence, the method comprising:
a step of providing two or more cells or cell-like structures;
a step of generating a small section containing one or more cells or cell-like structures, from the cells or cell-like structures;
a step of lysing the cells or cell-like structures contained in the small section such that the nucleic acids in the cells or cell-like structures are eluted and retained in the small section;
a step of bringing the nucleic acid into contact with a nucleic acid amplification reagent to amplify the nucleic acids in the small section to obtain an amplification product, as necessary;
a step of accommodating one or a plurality of the large sections including the small sections containing the amplification product, in a container for nucleotide sequencing, as necessary;
a step of determining a nucleotide sequence of the amplification product;
a step of analyzing the nucleotide sequence obtained in the step; and
a step of analyzing a region encoding genes in the nucleotide sequence to obtain a nucleic acid sequence encoding genes and/or an amino acid sequence encoded by the nucleic acid sequence.

49. A method for creating a database of sequences encoding genes in cells or cell-like structures, the method comprising:
a step of providing two or more cells or cell-like structures;
a step of generating a small section containing one or more cells or cell-like structures, from the cells or cell-like structures;
a step of lysing the cells or cell-like structures contained in the small section such that the nucleic acids in the cells or cell-like structures are eluted and retained in the small section;
a step of bringing the nucleic acid into contact with a nucleic acid amplification reagent to amplify the nucleic acids in the small section to obtain an amplification product, as necessary;
a step of accommodating one or a plurality of the large sections including the small sections containing the amplification product, in a container for nucleotide sequencing, as necessary;
a step of determining a nucleotide sequence of the amplification product;
a step of analyzing the nucleotide sequence obtained in the step;
a step of analyzing a region encoding genes in the nucleotide sequence to obtain a nucleic acid sequence encoding genes and/or an amino acid sequence encoded by the nucleic acid sequence; and
a step of creating a database of sequences encoding genes by using the nucleic acid sequence encoding genes and/or the amino acid sequence encoded by the nucleic acid sequence, and as necessary, information on cells or cell-like structures.

50. A method for generating a data structure that constitutes a sequence encoding genes in cells or cell-like structures, the method comprising:
a step of providing two or more cells or cell-like structures;
a step of generating a small section containing one or more cells or cell-like structures, from the cells or cell-like structures;
a step of lysing the cells or cell-like structures contained in the small section such that the nucleic acids in the cells or cell-like structures are eluted and retained in the small section;
a step of bringing the nucleic acid into contact with a nucleic acid amplification reagent to amplify the nucleic acids in the small section to obtain an amplification product, as necessary;
a step of accommodating one or a plurality of the large sections including the small sections containing the amplification product, in a container for nucleotide sequencing, as necessary;
a step of determining a nucleotide sequence of the amplification product;
a step of analyzing the nucleotide sequence obtained in the step;
a step of analyzing a region encoding a gene in the nucleotide sequence to obtain a nucleic acid sequence encoding genes and/or an amino acid sequence encoded by the nucleic acid sequence; and
a step of generating a data structure of sequences encoding genes, which are defined by the nucleic acid sequence encoding genes and/or the amino acid sequence encoded by the nucleic acid sequence, and as necessary, information on cells or cell-like structures.

51. A database generated by the method according to claim 49.

52. The database according to claim 50, wherein the database includes information indicating that the database is generated by the method according to claim 49.

53. The database according to claim 51 or 52, wherein the database contains full-length gene sequence percentage as a field.

54. The database according to any one of claims 51 to 53, wherein the database has a full-length gene sequence percentage of 20% or more for the nucleic acid sequence encoding contained genes and/or the amino acid sequence.

55. A data structure generated by the method according to claim 50.

56. The data structure according to claim 55, wherein the data structure includes information indicating that the data structure is generated by the method according to claim 50.

57. The data structure according to claim 55 or 56, wherein the data structure contains full-length gene sequence percentage as a field.

58. The data structure according to any one of claims 55 to 57, wherein the data structure has a full-length gene sequence percentage of 20% or more for the nucleic acid sequence encoding contained genes and/or the amino acid sequence.

59. The database or data structure according to any one of claims 51 to 58, wherein the database or data structure includes fields related to a coding sequence, and the fields related to a coding sequence are linked to contigs used in creating a genome database or a metagenome database.

60. The database or data structure according to claim 59, wherein the fields related to the coding sequences include a field that identifies whether the sequence is a full-length gene.

61. A method for analyzing an amino acid or nucleic acid sequence, comprising a step of analyzing the target amino acid or nucleic acid sequence using the nucleic acid sequence encoding genes or the amino acid sequence obtained by the method according to claim 48, the database generated by the method according to claim 49, the data structure generated by the method according to claim 50, the database according to any one of claims 51 to 54 and 57 to 60, or the data structure according to any one of claims 55 to 60.

62. The method for analyzing an amino acid or nucleic acid sequence according to claim 61, wherein the analyzing step includes binning contigs to generate a genomic or metagenome database.

63. A method for preserving nucleic acid molecules, comprising a step of further preserving a large section accommodating small sections as it is or after adding a substance inhibiting DNA degradation at room temperature or less, after the final step of the method for preparing a nucleic acid library according to claims 19 to 35.

64. The method according to claim 63, wherein the nucleic acid molecules are from microorganisms.

65. The method according to claim 63 or 64, wherein a temperature which is equal to or lower than room temperature is 4 °C or lower.

66. The method according to any one of claims 63 to 65, wherein a temperature which is equal to or lower than room temperature is −20 °C or lower.

67. The method according to any one of claims 63 to 66, wherein a temperature which is equal to or lower than room temperature is −80 °C or lower.
